(19) United States
(12) Patent Application Publication
(10) Pub. No.: US 2009/0012533 A1
(43) Pub. Date: Jan. 8, 2009

(54) Robotic instrument control system

(75) Inventors: Federico Barbagli, San Francisco, CA (US); Christopher R. Carlson, Menlo Park, CA (US)

Correspondence Address: David T. Burse;Vista IP Law Group LLP, Suite D-2, 12930 Saratoga Avenue, Saratoga, CA, 95070, US

(73) Assignee: Hansen Medical, Inc., Mountain View, CA (US)

(21) Appl. No.: 12/150,110

(22) Filed: Apr. 23, 2008

Related U.S. Application Data
(60) Provisional application No. 60/926,020, filed on Apr. 23, 2007.

(51) Int. Cl. A61B 19/00

(52) U.S. Cl. 606/130

(57) ABSTRACT

A robotic instrument system includes a controller configured to control actuation of at least one servo motor, and an elongate bendable guide instrument defining a lumen and operatively coupled to, and configured to move in response to actuation of, the at least one servo motor. The controller controls movement of the guide instrument via actuation of the at least one servo motor based at least in part upon a control model, wherein the control model takes into account an attribute of an elongate working instrument positioned in the guide instrument lumen.

### CROSS-REFERENCE TO RELATED APPLICATIONS

The present application claims the benefit under 35 U.S.C. §119 to U.S. Provisional Application No. 60/926,020, filed Apr. 23, 2007, the contents of which are incorporated herein by reference as though set forth in full.

The present application may also be related to subject matter disclosed in the following applications and patents, the contents of which are also incorporated herein by reference as though set forth in full: U.S. patent application Ser. Nos. 10/923,660, filed Aug. 20, 2004; 10/949,032, filed Sep. 24, 2005; 11/073,363, filed Mar. 4, 2005; 11/173,812, filed Jul. 1, 2005; 11/176,954, Jul. 6, 2005; 11/179,007, Jul. 6, 2005; 11/185,432, filed Jul. 19, 2005; 11/202,925, Aug. 12, 2005; 11/331,576, filed Jan. 13, 2006; 11/418,398, filed May 3, 2006; 11/481,433, filed Jul. 3, 2006; 11/637,951, filed Dec. 11, 2006; 11/640,099, filed Dec. 14, 2006; 60/879,911, filed Jan. 10, 2007; 11/678,016, filed Feb. 22, 2007 and U.S. Provisional Application Nos. 60/750,590, filed Dec. 14, 2005; 60/756,136, filed Jan. 3, 2006; 60/776,065, Feb. 22, 2006; 60/785,001, filed Mar. 22, 2006; 60/788,176, filed Mar. 31, 2006; 60/801,355, filed May 17, 2006; 60/801,546, filed May 17, 2006; 60/801,945, filed May 18, 2006; 60/833,624, filed Jul. 26, 2006; 60/835,592, filed Aug. 3, 2006; 60/838,075, filed Aug. 15, 2006; 60/840,331, filed Aug. 24, 2006; 60/843,274, filed Sep. 8, 2006; 60/873,901, filed Dec. 8, 2006; 60/899,048, filed Feb. 1, 2007; 60/900,584, filed Feb. 8, 2007; U.S. Provisional Patent Application No. 60/902,144, filed Feb. 15, 2007.

### FIELD OF THE INVENTION

The invention relates generally to robotically controlled systems, such as tele-robotic surgical systems, and more particularly, to a robotic catheter system for performing minimally invasive diagnostic and therapeutic procedures.

### BACKGROUND

Robotic interventional systems and devices are well suited for performing minimally invasive medical procedures as opposed to conventional techniques wherein the patient's body cavity is open to permit the surgeon's hands access to internal organs. Traditionally, surgery utilizing conventional procedures meant significant pain, long recovery times, lengthy work absences, and visible scarring. However, advances in technology have lead to significant changes in the field of medical surgery such that less invasive surgical procedures, in particular, minimally invasive surgery (MIS), are increasingly popular.

A “minimally invasive medical procedure” is generally defined as a procedure that is performed by entering the body through the skin, a body cavity, or an anatomical opening utilizing small incisions rather than large, open incisions in the body. Various medical procedures are considered to be minimally invasive including, for example, mitral and tricuspid valve procedures, patent formen ovale, atrial septal defect surgery, colon and rectal surgery, laparoscopic appendectomy, laparoscopic esophagectomy, laparoscopic hysterectomies, carotid angioplasty, vertebroplasty, endoscopic sinus surgery, thoracic surgery, donor nephrectomy, hypodermic injection, air-pressure injection, subdermal implants, endoscopy, percutaneous surgery, laparoscopic surgery, arthroscopic surgery, cryosurgery, microsurgery, biopsies, videoscope procedures, keyhole surgery, endovascular surgery, coronary catheterization, permanent spinal and brain electrodes, stereotactic surgery, and radioactivity-based medical imaging methods. With MIS, it is possible to achieve less operative trauma for the patient, reduced hospitalization time, less pain and scarring, reduced incidence of complications related to surgical trauma, lower costs, and a speedier recovery.

Special medical equipment may be used to perform MIS procedures. Typically, a surgeon inserts small tubes or ports into a patient and uses endoscopes or laparoscopes having a fiber optic camera, light source, or miniaturized surgical instruments. Without a traditional large and invasive incision, the surgeon is not able to see directly into the patient. Thus, the video camera serves as the surgeon's eyes. The images of the interior of the body are transmitted to an external video monitor to allow a surgeon to analyze the images, make a diagnosis, visually identify internal features, and perform surgical procedures based on the images presented on the monitor.

MIS procedures may involve minor surgery as well as more complex operations that involve robotic and computer technologies, which may be used during more complex surgical procedures and have led to improved visual magnification, electromechanical stabilization, and reduced number of incisions. The integration of robotic technologies with surgeon skill into surgical robotics enables surgeons to perform surgical procedures in new and more effective ways. Although MIS techniques have advanced, physical limitations of certain types of medical equipment still have shortcomings and can be improved. While known devices may have been used effectively, they may lack the required or desired control over system components that manipulate and position a working instrument.

For example, various working instruments in the form of catheters, e.g., ablation catheters, may be robotically controlled. Different ablation catheters may have different mechanical and physical attributes and characteristics. In some cases, this is true of catheters that are the same type and made by the same manufacturer, e.g., due to variations during the manufacturing process. For example, the outer diameters of two catheters of the same type may vary slightly. Further, different catheters made by different manufacturers may have different mechanical and physical attributes. For example, different components may have different shapes, dimensions, different stiffness or modulus attributes, etc., resulting in different extension, retraction and bending compared to what is expected or desired when a control model is executed. Known robotic surgical systems, however, do not account for these mechanical and/or structural differences or variances. Rather, for example, control models of known robotic surgical systems are based on an assumption that certain mechanical and/or physical attributes of certain working instruments are the same such that the same control model is applied. As a result, with known systems, the same control model may be applied to two catheters despite the catheters having different mechanical and/or physical properties or attributes that may cause execution of the control model to manipulate the two catheters in different ways, thereby resulting in positioning errors, which may be minor or significant depending on the circumstances and system configuration.

For example, the same robotic guide catheter is likely to perform differently with a relatively stiff grasping mechanism placed through the working lumen, as opposed to a very thin, very bendable light transmitting fiber. Further, two working instruments in the form of ablation catheters may have similar, but different, outer diameters. As a result, larger frictional forces may exist between an outer surface of the larger ablation catheter and an inner surface of the guide catheter. These larger frictional forces may result in reduced extension or maneuverability of the ablation catheter than what is called for by a control model. As a result, a surgeon and/or robotic surgical system may believe that the distal end of the ablation catheter is extended and shaped to assume a desired position when in fact the ablation catheter has not reached the desired position due to the increased frictional force.

As another example, one ablation catheter may be stiffer or less susceptible to bending than another ablation catheter. Thus, in order to properly position the stiffer ablation catheter at a certain angle, a larger amount of force must be applied. However, with a fixed control model, the same amount of force may be applied to each catheter, resulting in one catheter bending less than the other, thereby resulting in possible positioning errors. Similar issues may arise in cases in which a catheter is more bendable in one plane compared to another plane.

In some cases, these errors may be small, but even small errors may impact the effectiveness of a control model and how accurately a working instrument can be manipulated, particularly considering that a robotic surgical system must often traverse a number of vascular curves. Consequently, control, manipulation and positioning of a working instrument or tool may be difficult with known surgical systems, thereby resulting in more complicated and/or less effective procedures.

### SUMMARY

One embodiment is directed to a robotic instrument system comprising a controller and an elongate bendable guide instrument. The controller is configured to control actuation of at least one servo motor. The guide instrument defines a lumen and is operatively coupled to, and configured to move in response to actuation of, the servo motor. The controller controls movement of the guide instrument via actuation of the at least one servo motor based at least in part upon a control model, which takes into account an attribute of an elongate working instrument positioned in the guide instrument lumen.

Another embodiment is directed to a robotic instrument system that comprises a controller, an instrument driver and an elongate flexible guide instrument. The instrument driver is in communication with the controller and has an instrument interface including an instrument drive element that moves in response to control signals generated by the controller. The guide instrument has a base and a distal bending portion. The base is operatively coupled to the instrument interface. The guide instrument includes a control element having first and second end portions. The first end portion is operatively coupled to the instrument drive element through the base, and the second end portion is coupled to the distal bending portion. The control element is axially moveable relative to the guide instrument by movement of the instrument drive element. The controller implements a desired bending of the distal bending portion of the guide instrument by selected movement of the instrument drive element based at least in part on a control model, which takes into account one or both of a mechanical attribute and a physical attribute of an elongate working instrument that is positioned within the distal bending portion of the guide instrument.

According to another embodiment, a robotically controlled medical instrument system comprises a controller, an instrument driver, a guide instrument and a working instrument. The instrument driver is operatively coupled to the controller and controllable according to a control model employed by the controller. The guide instrument is operatively coupled to the instrument driver and comprises at least one wire extending there through for controllably articulating a distal bending portion of the guide instrument under control of the instrument driver. The working instrument is positioned in a working lumen of the guide instrument and at least partially extends through the distal bending portion. The controller is adapted to automatically adjust the control model based on an attribute of the working instrument.

In one or more embodiments, the attribute is a mechanical or physical attribute of a portion of the working instrument positioned within a distal bending portion of the guide instrument. For example, the attribute of the working instrument may be mechanical impedance, a stiffness, or a modulus of the working instrument. Moreover, the control model takes into account a frictional force between an outer surface of working instrument and an inner surface of the guide instrument. Additionally, the control model may also take into account one or both of a type and size of the working instrument. Further, the control model may be adapted to take into account a working instrument having sections comprising differing dimensions or other physical attributes.

In one or more embodiments, the control model is a kinematic model. The kinematic model may be based in part upon a mechanical parameter of the guide instrument. The kinematic model may be utilized by the controller to determine a movement of the instrument drive element based upon a relationship between an angular rotation of the drive element and a resulting position of the distal bending portion of the guide instrument. In one embodiment, a control model comprises a forward kinematics model expressing a desired position of a distal end portion of the guide instrument as a function of actuated inputs for controlling a control element of the guide instrument, and an inverse kinematics model expressing actuated inputs for controlling the control element of the guide instrument as a function of the desired position of the distal end portion of the guide instrument.

In one or more embodiments, the controller is configured to obtain the attribute of the working instrument from a data storage element attached to or associated with the working instrument.

### BRIEF DESCRIPTION OF THE DRAWINGS

Referring now to the drawings in which like reference numbers represent corresponding parts throughout and in which:

FIG. 1 is a block diagram of a system constructed according to one embodiment for accounting for the particular working instrument employed in a robotic instrument system;

FIG. 2 is a flow chart of a method of accounting for the particular working instrument employed in a robotic instrument system according to one embodiment;

FIG. 3 illustrates a robotic surgical system in which apparatus and method embodiments may be implemented;

FIG. 4 further illustrates coaxial sheath and guide catheter instruments and a working instrument positioned within a working lumen of the guide catheter of the system shown in FIG. 3;

FIG. 5 illustrates an example of an operator workstation of the robotic surgical system shown in FIG. 3 with which a catheter instrument can be manipulated using different user interfaces and controls;

FIG. 6 further illustrates a control system for use with the robotic surgical system shown in FIG. 3;

FIG. 7 illustrates a support assembly or mounting brace for a instrument driver of the robotic surgical system shown in FIG. 3;

FIG. 8 illustrates the support assembly shown in FIG. 7 in greater detail;

FIG. 9 is a perspective view of an instrument driver to which sheath and guide catheter instruments may be mounted for use in the system shown in FIG. 3;

FIG. 10 illustrates sheath and guide catheter instruments coupled to respective mounting plates of an instrument driver for use in the system shown in FIG. 3;

FIG. 11 is a perspective view of a catheter instrument that may be used in a robotic surgical system;

FIG. 12 is a perspective view of a coaxial guide/sheath catheter instrument that may be used in a robotic surgical system;

FIGS. 13A-16B are respective perspective and cross-sectional views of a catheter and controllable bending thereof by manipulation of a control element;

FIGS. 17-22 illustrate software control schema in accordance with various embodiments;

FIG. 23 illustrates a kinematics control model utilizing forward kinematics and inverse kinematics;

FIG. 24 illustrates task coordinates, joint coordinates, and actuation coordinates of a kinematics model;

FIG. 25 illustrates variables of a kinematics model associated with a geometry of a catheter;

FIG. 26 illustrates a method for generating a haptic signal;

FIG. 27 illustrates a method for converting an operator hand motion to a catheter motion utilizing a kinematics model;

FIG. 28 represents an operation of components of an instrument driver;

FIG. 29 illustrates a set of equations associated with the diagram of FIG. 28;

FIGS. 30-33 illustrate equations associated with an operation of a guide instrument interface socket in accordance with some embodiments;

FIG. 34 is a flow chart of a method of controlling a robotic instrument system according to another embodiment based on mechanical and/or physical data of a working instrument;

FIG. 35 is a flow chart of a method of controlling a robotic instrument system according to another embodiment based on mechanical and/or physical data of a working instrument and adjusting a coefficient and/or variable of a control model;

FIG. 36 illustrates a lookup table or database including physical data of a working instrument that includes outer diameter dimensions that are used to adjust a control model and account for the particular working instrument employed;

FIG. 37 illustrates a lookup table or database including mechanical data of a working instrument in the form of friction forces between an outer surface of the working instrument and an inner surface of a guide catheter and corresponding control model adjustments that are used to account for the working instrument employed;

FIG. 38 illustrates a lookup table or database including mechanical data of a working instrument in the form of stiffness or modulus values and corresponding control model adjustments that are used to account for the working instrument employed;

FIG. 39 illustrates a lookup table or database including mechanical data of a working instrument in the form of stiffness or modulus values of different types of working instruments and corresponding control model adjustments that are used to account for the particular working instrument employed;

FIG. 40 is a block diagram of a system constructed according to another embodiment that includes a database of a plurality of control models corresponding to different working instruments that may be employed with a robotic instrument system; and

FIG. 41 illustrates one example of a database for selecting a control model to account for the particular working instrument employed.

### DETAILED DESCRIPTION OF ILLUSTRATED EMBODIMENTS

Embodiments are directed to systems and methods for controlling a robotic instrument system by selecting a specific control model from multiple control models that correspond to different working instruments and/or attributes thereof, or adapting or adjusting a control model based on one or more attributes of the working instrument. The selected or adjusted control model is used to manipulate and position a working instrument or tool, such as an ablation catheter, at a desired position and orientation within a patient, e.g., through the vasculature of the patient to treat cardiac tissue. The control model that is selected or adjusted advantageously accounts for the particular working instrument that is employed. More specifically, embodiments advantageously account for mechanical and/or physical differences between working instruments that may be of the same type and made by the same manufacturer, working instruments that may be of the same type and made by different manufacturers, and different types of working instruments. In this manner, a control model of the robotic system is selected or adjusted as necessary to compensate for variations resulting from a particular working instrument that may otherwise cause positioning discrepancies or errors when a general, all-purpose control model is used as in known robotic instrument systems. Thus, embodiments provide more accurate control over manipulation and positioning of the working instrument and the effectiveness of the surgical procedure.

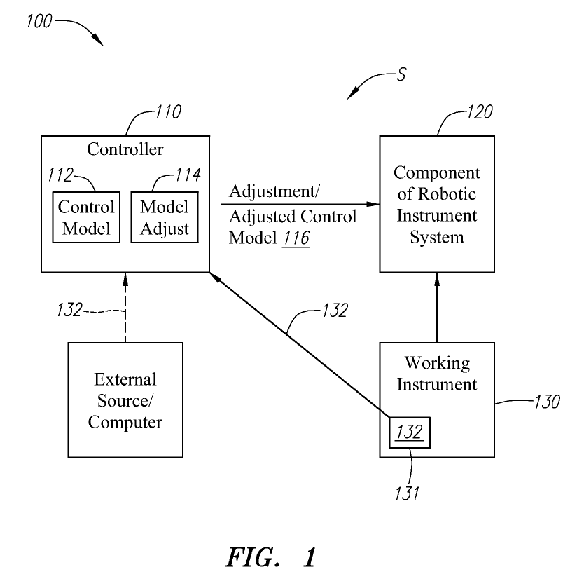

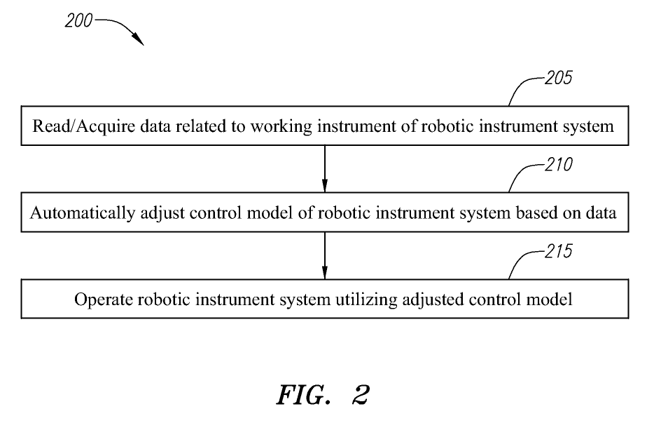

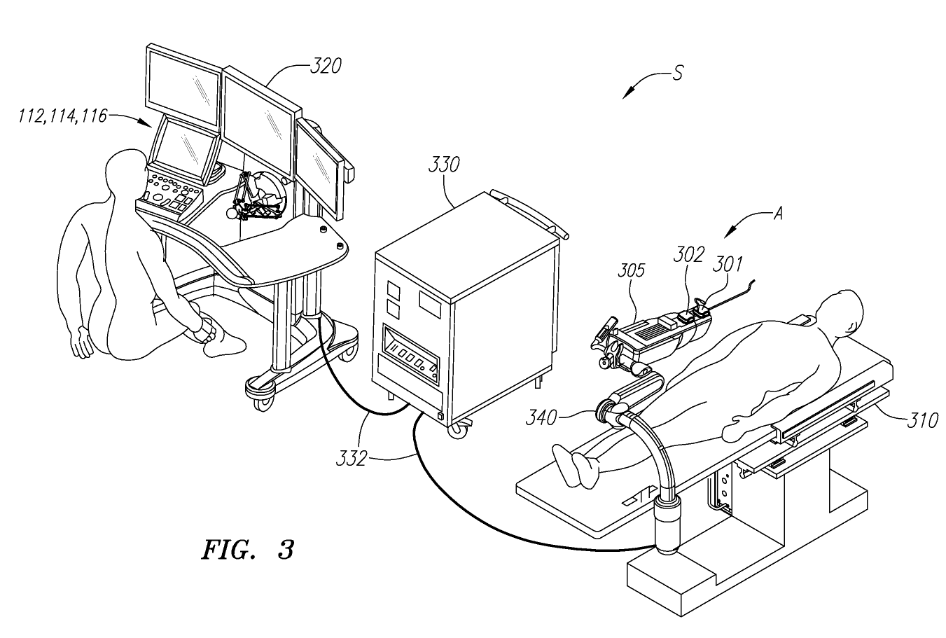

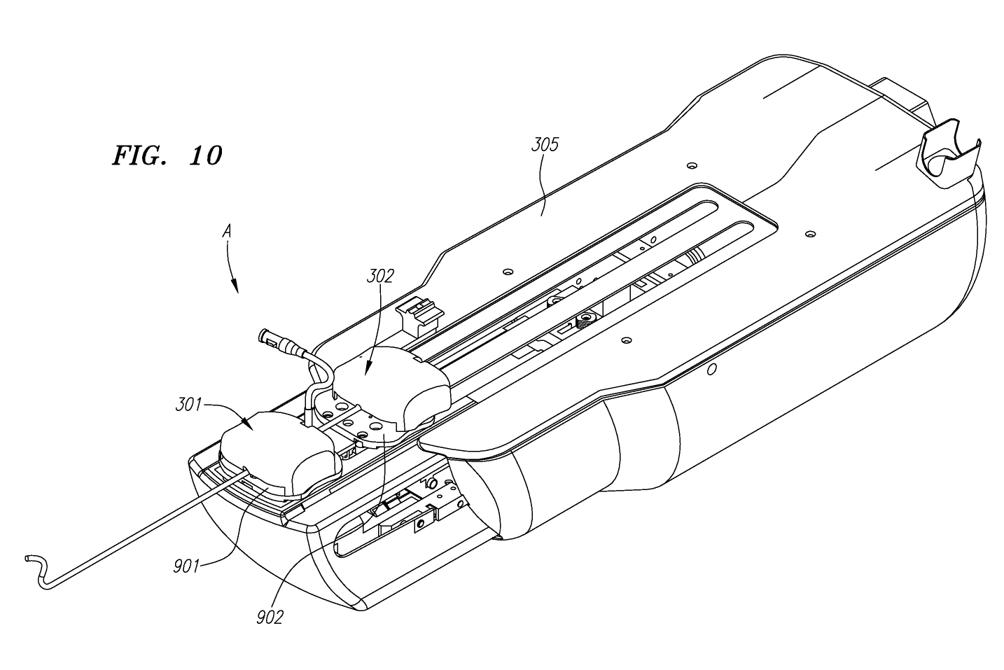

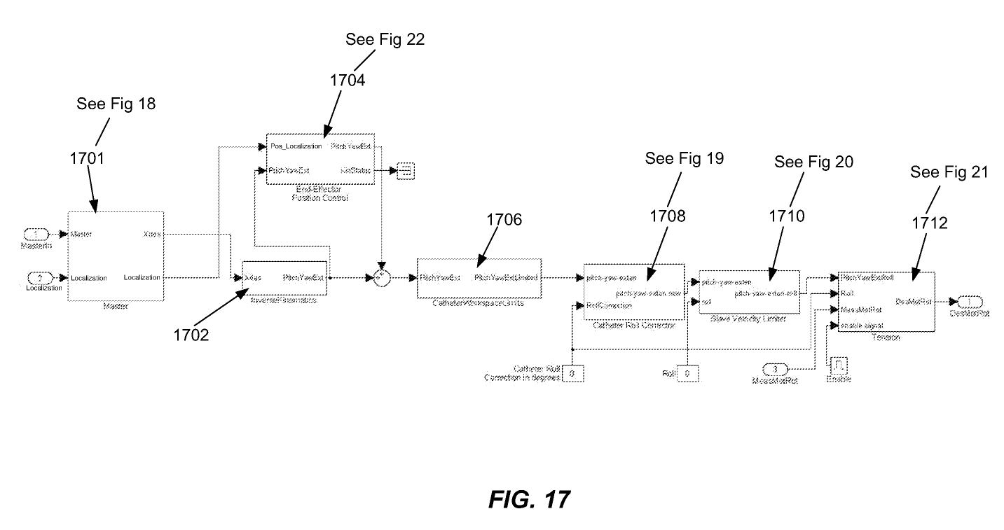

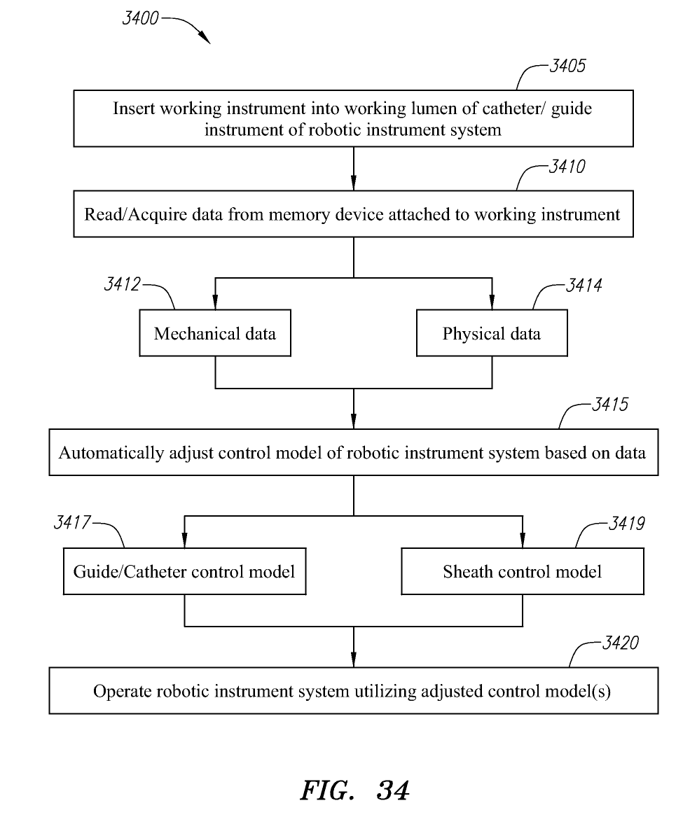

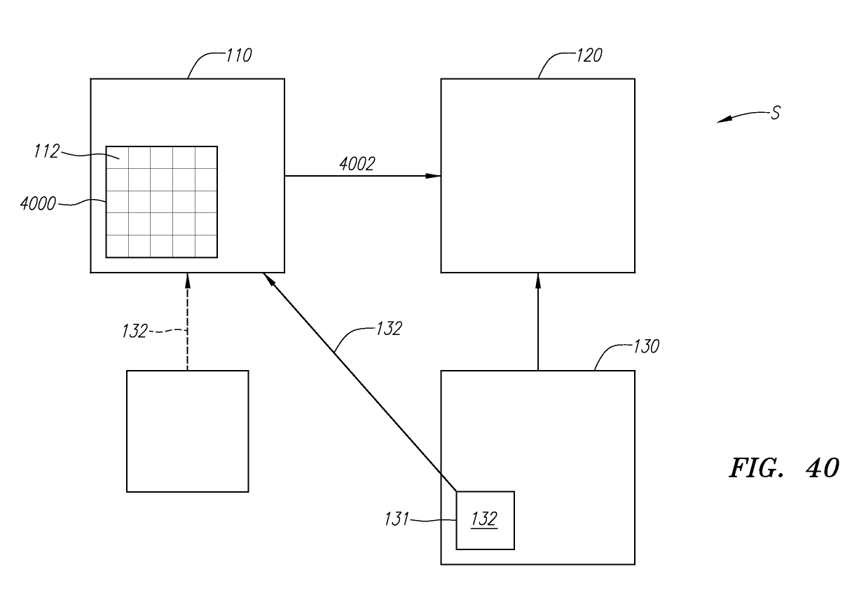

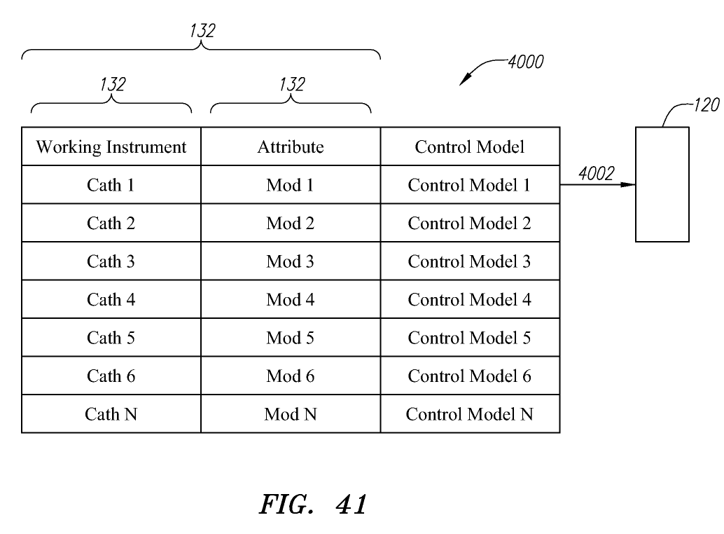

FIG. 1 is a block diagram of a system constructed according to one embodiment for adjusting or adapting a control model of a robotically controlled surgical system to account for the particular working instrument employed in the robotic surgical system. FIG. 2 is a flow diagram of a method of adjusting a control model for a given working instrument according to one embodiment. FIGS. 3-16B illustrate in further detail one example of a robotic surgical system and components thereof in which embodiments of the invention may be implemented. FIGS. 17-33 illustrate in further detail examples of components of a robotic surgical system and a kinematics control model 112 that can be adjusted 114 for a particular working instrument 130 according to embodiments of the invention. FIGS. 34-39 illustrate methods and flow charts or databases for adjusting or adapting a control model of a robotically controlled surgical system according to other embodiments and that can be implemented in the system described with reference to FIGS. 1-33. FIGS. 40-41 illustrate system and method embodiments for selecting a control model from a database of a plurality of control models that are pre-programmed or already configured to account for the particular working instrument employed.

Referring to FIG. 1, a system 100 constructed according to one embodiment includes robotic surgical system S that includes a controller 110 and one or more instrument components 120, such as a sheath instrument and a guide catheter instrument (described in further detail with reference to FIGS. 3-33) through which a working instrument or tool 130 (generally working instrument 130) is inserted. The controller 110 includes one or more control models 112 that may be implemented in software, hardware, or a combination thereof to control and manipulate one or more system components 120 in order to manipulate and position the working instrument 130 disposed therein. In known systems, the control model 112 is applied to various working instruments 130.

According to one embodiment, the controller 110 or other associated storage device or control element includes a control model adjustment 114, which may also be implemented in software, hardware or a combination thereof. The control model adjustment 114 modifies, adapts or adjusts the standard control model 112 depending on the particular working instrument 130 that is utilized. In one embodiment, the control model 112 is adjusted to account for specific physical and/or mechanical properties of the working instrument 130.

For this purpose, in one embodiment, the working instrument 130 includes a memory or data storage device 132, which may be attached to, carried by or otherwise associated with the working instrument 130. The controller 110 is configured to read, receive or acquire the data 132 from the storage device 131, e.g., after the working instrument 130 is inserted within a guide catheter instrument component 120 of the system. In another embodiment, the data 132 can be entered through, or read from, an external source or computer 140, either automatically or with manual input by an operator. For ease of reference, embodiments are described with reference to a working instrument 130 that includes a data storage device 131, and a controller 110 that acquires working instrument data 132 utilizing suitable known electrical, optical and/or wireless communications (e.g., as a RFID device).

According to one embodiment, the working instrument 130 is an ablation catheter. According to another embodiment, the working instrument 130 is a needle. In another embodiment, the working instrument 130 is a dilator. In a further alternative embodiment, the working instrument 130 is a biopsy forceps. For ease of explanation, reference is made to a working instrument 130 generally or to a working instrument 130 in the form of an ablation catheter, but it should be understood that embodiments can be implemented using different types of working instruments 130 including those mentioned above. Moreover, embodiments can be implemented using working instruments 130 that are from the same or different suppliers or manufacturers. Further, in another embodiment, the working instruments 130 are of the same type (e.g. ablation catheters), but from the same suppliers. Further, the working instruments 130 may be of the same type and from the same manufacturer. Embodiments can also be implemented using different types of working instruments 130, e.g., a combination of one or more ablation catheters and another type of working instrument 130.

Referring to FIG. 2, a method 200 for controlling a robotic surgical system according to one embodiment utilizing the system 100 shown in FIG. 1 includes acquiring or reading data 132 of a mechanical and/or physical attribute of a particular working instrument 130 that is a part of or utilized with the robotic instrument system at step 205. At step 210, at least one control model 112 is adjusted 114 for the particular working instrument 130 that is utilized based on the acquired data 132. According to one embodiment, the adjustment 114 is performed automatically, e.g., by the controller 110 or another control component. One or more robotic surgical system S components may then be controlled using an adjusted control model or adjusted model parameter 116 (generally referred to as adjusted control model) that is adapted for the particular working instrument 130 to account for the unique mechanical and/or physical attributes or properties of the working instrument 130.

According to one embodiment, the data 132 acquired is data of a physical attribute of the working instrument 130, such as the outer diameter or width of a bendable or working distal portion of an ablation catheter 130. Embodiments advantageously account for these different dimensions and associated different friction forces resulting from these variances, even for ablation catheters 130 of the same type. More particularly, two ablation catheters 130 that may be used with the system may have similar dimensions, but the dimensions may nevertheless vary. These variances may occur, for example, with the same type of ablation catheters 130, catheters 130 from the same manufacturer, and catheters 130 from different manufacturers. Utilizing a wider ablation catheter 130 may result in larger frictional forces between an outer surface of the catheter 130 and an inner surface of a system component 120, e.g., an inner surface of a guide catheter through which the ablation catheter 130 is inserted. This larger frictional force may impact the manner in which the ablation catheter 130 extends from, or retracts into, the guide catheter, and the manner in which the guide catheter and ablation catheter 130 traverse vascular curvature.

With embodiments, the control model 112 of the guide catheter component 120 is advantageously adjusted 114 to account for these different diameters or widths, even if the difference is small, to generate a an adjusted or modified control model 116 that can be executed to achieve the desired guide catheter manipulation, regardless of whether the smaller or larger ablation catheter 130 is utilized, such that the ablation catheter 130 is manipulated and positioned as desired.

In one embodiment, as discussed above, the physical attribute is the outer diameter of the working or bendable portion of the working instrument 130, which may have a substantially consistent width or diameter that nevertheless varies to a certain degree to result in a discrepancy between the expected or desired position and the actual position of the working instrument 130. This discrepancy can be compensated using an adjusted control model 116 even through such variation may not be visible by a human eye. In another embodiment, the bendable or working portion of the working instrument 130 has a plurality of segments having different widths or diameters. The control model 112 can be adjusted 114 to account for different segments that may impact operability of one or more components 120. For example, the working instrument 130 may be at a first position such that a first segment, e.g., a wider segment, has a larger impact on the components 120 (due to larger friction forces), whereas when the working instrument 130 is at a second, more distal position, a second segment, e.g., a narrower segment, has a larger impact. The control model 112 can be adjusted 114 to account for different segments of the working instrument 130 that may have a larger impact on the manipulation and control of system components 120 compared to other segments.

According to another alternative embodiment, the data 132 is data of a mechanical attribute of the working instrument 130. In one embodiment, the data 132 is a stiffness of the working instrument 130, e.g. represented by a modulus value such as Young's Modulus. For example, a stiffer ablation catheter 130 will require more force to achieve a desired bend compared to a more flexible ablation catheter 130. However, using the same control model 112 for ablation catheters 130 having different stiffness attributes or modulus values results in bending one ablation catheter 130 more than the other, resulting in positioning errors. Embodiments advantageously adjust 114 the control model 112 such that the same or substantially similar bending may be achieved using ablation catheters 130 having different stiffness or modulus values. According to another alternative embodiment, the data 132 is a mechanical impedance of the working instrument 130. According to a further embodiment, the data 132 includes both mechanical and physical attributes of a working instrument 130.

FIGS. 3-16B illustrate in further detail one example of a robotic surgical system S and components thereof in which embodiments of the invention, including the embodiments shown in FIGS. 1-2, may be implemented. In the illustrated example, the system S includes a robotic catheter assembly A having a robotic or first or outer steerable complement, otherwise referred to as a sheath instrument 301 (generally referred to as a “sheath” or a “sheath instrument”) and/or a second or inner steerable component, otherwise referred to as a robotic catheter or guide or catheter instrument 302 (generally referred to as a “guide catheter” or a “catheter instrument”). The sheath 301 and guide catheter 302 are controllable using a robotic instrument driver 305 (generally referred to as “instrument driver”). During use, a patient is positioned on an operating table or surgical bed 310 (generally referred to as “operating table”) to which a robotic catheter assembly A is coupled or mounted. In the illustrated example, the system S includes an operator workstation 320, an electronics rack 330 and associated bedside electronics box, a setup joint mounting brace 340, and the instrument driver 305. A surgeon is seated at the operator workstation 320 and can monitor the surgical procedure, patient vitals, and control one or more catheter devices.

Various system S components in which embodiments of the invention may be implemented are illustrated in close proximity to each other in FIG. 1, but embodiments may also be implemented in systems (S) in which components are separated from each other, e.g., located in separate rooms. For example, the instrument driver 305, operating table 310, and bedside electronics box may be located in the surgical area with the patient, and the operator workstation 320 and the electronics rack 330 may be located outside of the surgical area and behind a shielded partition. System (S) components may also communicate with other system (S) components via a network to allow for remote surgical procedures during which the surgeon may be located at a different location, e.g., in a different building or at a different hospital utilizing a communication link transfers signals between the operator control station 320 and the instrument driver 305. System (S) components may also be coupled together via a plurality of cables or other suitable connectors 332 to provide for data communication, or one or more components may be equipped with wireless communication components to reduce or eliminate cables 332. In this manner, a surgeon or other operator may control a surgical instrument while being located away from or remotely from radiation sources, thereby decreasing the operator's exposure to radiation.

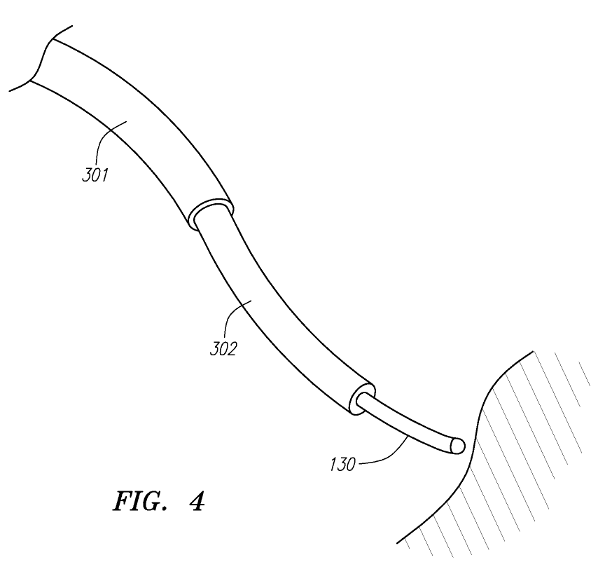

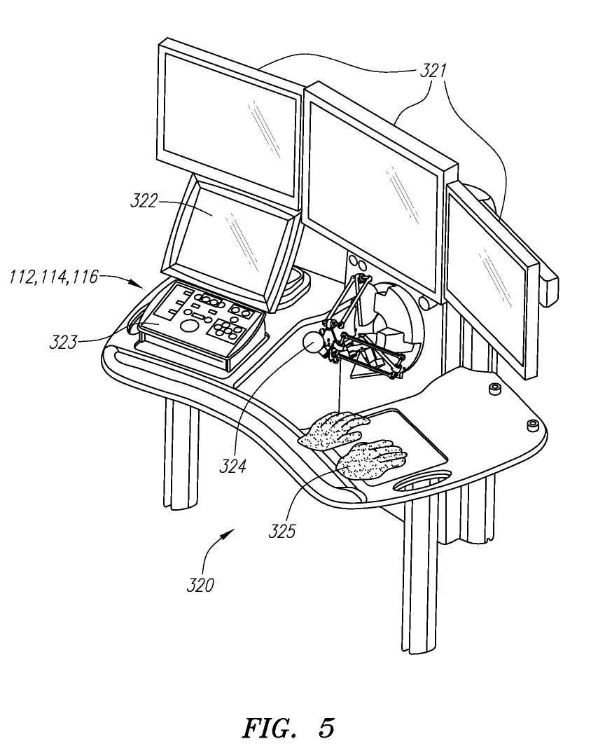

Referring to FIGS. 4-5, the operator workstation 320 according to one embodiment includes three display screens 321, a touchscreen user interface 322, a control button console or pendant 323, and a master input device (MID) 324. By manipulating the console 323 and MID 324, an operator can cause an instrument driver 305 to remotely control flexible guide and guide catheter instruments 301, 302 mounted to the instrument driver 305 and a working instrument 130 inserted through and disposed within the guide catheter 302, which may engage tissue (as shown in FIG. 4). The operator control station may be located away from radiation sources, thereby advantageously decreasing the operator's exposure to radiation.

Using the operator workstation 320, inputs to control a flexible catheter assembly (A) can entered using the MID 324 and data gloves 325, which serve as user interfaces through which the operator may control the instrument driver 305 and any instruments attached thereto. The instrument driver 305 and associated instruments may be controlled via manipulation of the MID 324, gloves 325, or a combination of both. The MID 324 may have integrated haptics capability for providing tactile feedback to the operator. It should be understood that while an operator may robotically control one or more flexible catheter devices via an inputs device, in one or more embodiments, a computer of the robotic catheter system may be activated to automatically position a catheter instrument and/or its distal extremity inside a patient or to automatically navigate the patient anatomy to a designated surgical site or region of interest.

The MID 325 software may be a proprietary module packaged with an off-the-shelf MID system, such as the Phantoms from SensAble Technologies, Inc., which is configured to communicate with the Phantoms Haptic Device hardware at a relatively high frequency as prescribed by the manufacturer. Other suitable MIDs 324 are available from suppliers such as Force Dimension of Lausanne, Switzerland.

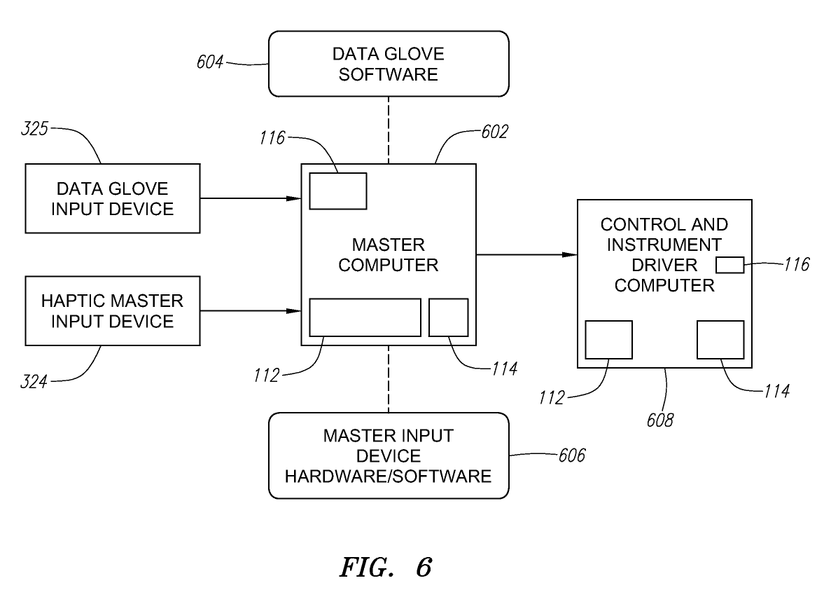

FIG. 6 is a block diagram illustrating an example system architecture in which embodiments may be implemented. In the illustrated example, a master computer 602 oversees the operation of the system (S) and is coupled to receive user input from hardware input devices such as a data glove input device 325 and MID 324. In the illustrated embodiment, the control model 112, control model adjustment 114 and/or modified control model 116 may be stored in or implemented in the master computer 602 as software, hardware, or a combination thereof.

The master computer 602 executes master input device software, data glove software, visualization software, instrument localization software, and software to interface with operator control station buttons and/or switches is depicted. Data glove software 604 processes data from the data glove input device 325, and MID hardware and software 606 processes data from the haptic MID 325. In response to the processed inputs, and in accordance with the adjusted control model 116, the master computer 602 processes instructions to instrument driver computer 608 to activate the appropriate mechanical response from the associated motors and mechanical components to achieve the desired response from the flexible catheter assembly (A). The control model 112, model adjustments 114, and/or adjusted control model 116 may also be stored in the instrument driver computer 608 and/or in another control element or computer as necessary and depending on the system architecture.

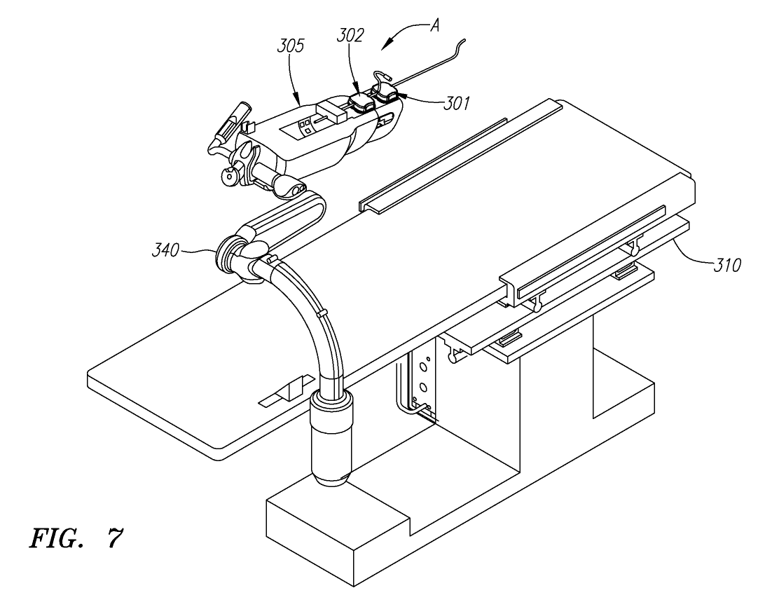

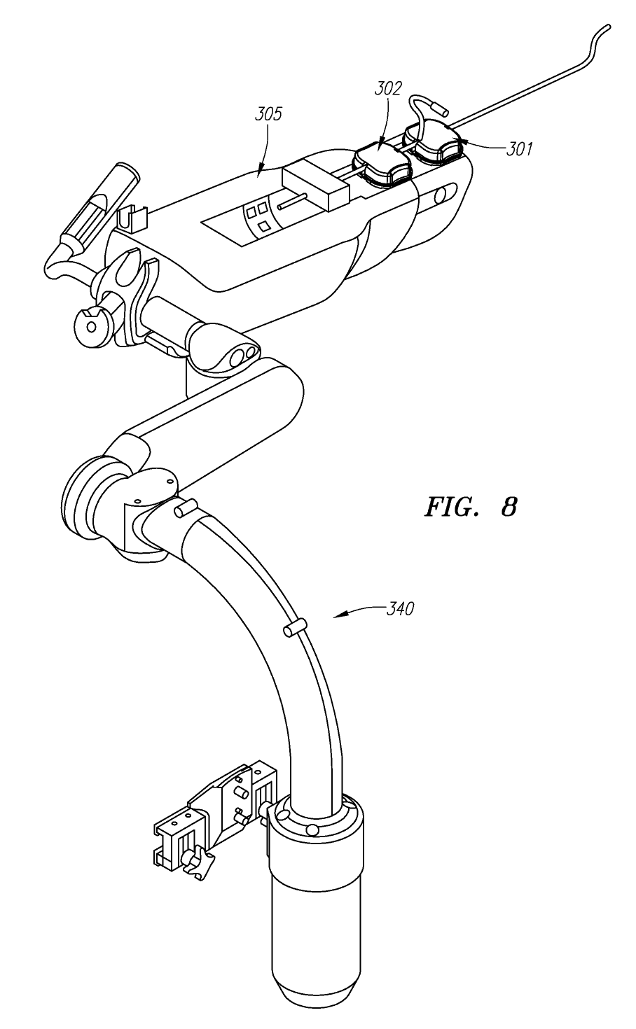

Referring to FIGS. 7-8, a system (S) includes a setup joint or support assembly 340 (generally referred to as “support assembly”) for supporting or carrying the instrument driver 305 over the operating table 310. One suitable support assembly 340 has an arcuate shape and is configured to position the instrument driver 305 above a patient lying on the table 310. The support assembly 340 may be configured to movably support the instrument driver 305 and to allow convenient access to a desired location relative to the patient. The support assembly 305 may also be configured to lock the instrument driver 305 into a certain position. In the illustrated example, the support assembly 340 is mounted to an edge of the operating table 310 such that sheath and catheter instruments 301, 302 mounted on the instrument driver 305 can be positioned for insertion into a patient. The instrument driver 305 is controllable to maneuver the catheter and/or sheath instruments 302, 301 within the patient during a surgical procedure. Although the figures illustrate a single guide catheter 302 and sheath 301 mounted on a single instrument driver 305, embodiments may be implemented in systems (S) having other configurations. For example, embodiments may be implemented in systems (S) that include a plurality of instrument drivers 305 on which a plurality of catheter/sheath instruments 302, 301 can be controlled. Further aspects of a suitable support assembly 340 are described in U.S. patent application Ser. No. 11/481,433 and U.S. Provisional Patent Application No. 60/879,911, the contents of which were previously incorporated herein by reference.

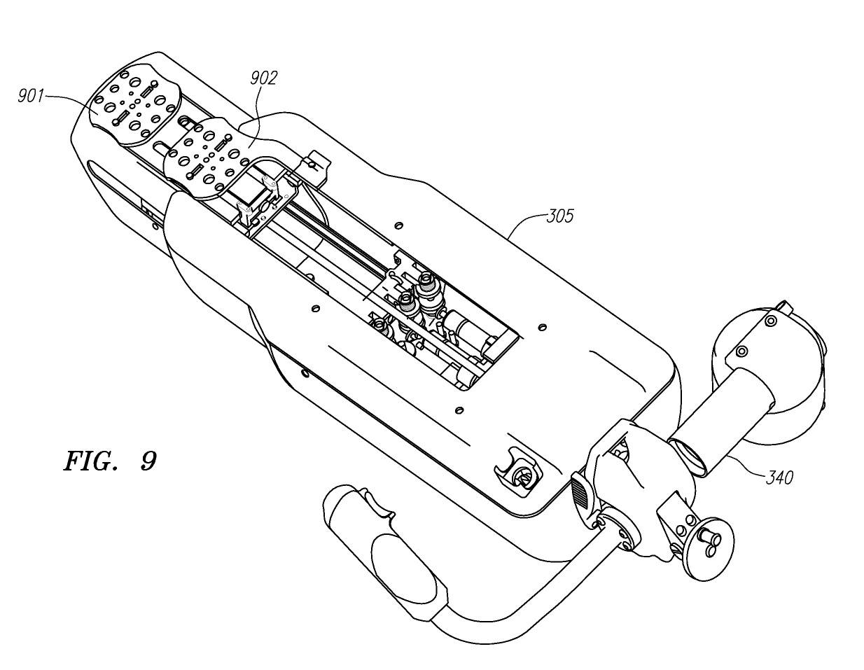

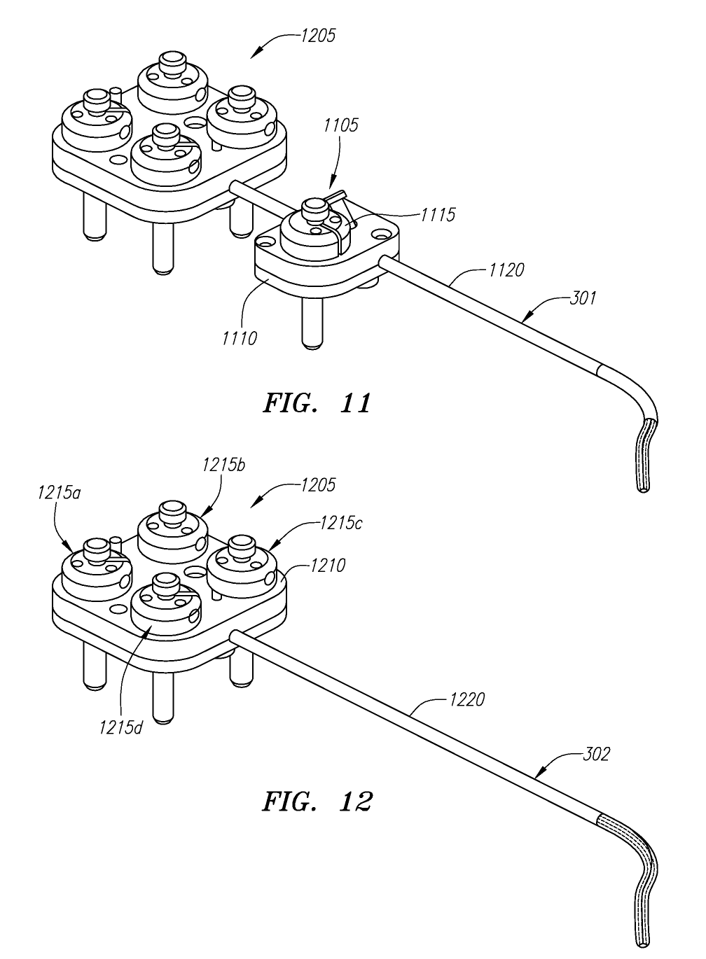

Referring to FIGS. 9-12, an instrument assembly (A) comprised of a sheath instrument 301 and an associated guide or catheter instrument 302 is mounted to associated mounting plates 901, 902 on a top portion of the instrument driver 305. During use, the guide catheter 302 is inserted within a central lumen of the sheath instrument 301 such that the guide 302 and sheath 301 are arranged in a coaxial manner. Although the instruments 301, 302 are arranged coaxially, movement of each instrument 301, 302 can be controlled and manipulated independently according to independent control models and servo motors of the instrument driver 305. For this purpose, motors within the instrument driver 305 (as shown in FIGS. 11-12) are controlled such that carriages coupled to the mounting plates 901, 902 are driven forwards and backwards on bearings. One or more components, such as the instrument driver 305, may also be rotated about a shaft to impart rotational motion to the guide catheter 302 and/or the sheath 301. As a result, the guide catheter 302 and the sheath instrument 301 can be controllably manipulated and inserted into and removed from the patient. Working instruments or tools 130 extending through the working lumen of the guide catheter 302 can also be controllably manipulated, bent and positioned as necessary.

In the illustrated example, the guide catheter 302 is coaxially disposed within the sheath 301, and is independently controllable relative to the sheath 301. As shown in FIG. 11, sheath instrument 301 includes a drivable assembly 1105, which includes an instrument base 1110 and a single control element interface assembly 1115, a sheath catheter member 1120, the proximal end of which is mounted within the instrument base 1110, and a control or tension element, such as a cable (not shown in FIG. 11) extending within the sheath catheter member 1120 and coupled to the interface assembly 1115, such that operation of the interface assembly 1115 bends the distal end of the sheath catheter member 1120 in one direction.

As shown in FIG. 12, the guide catheter 302 generally comprises a proximal drivable assembly 1205, which includes an instrument base 1210 and four control element interface assemblies 1215a-d, a catheter member 1220, the proximal end of which is mounted within the instrument base 1205, and four control or tension elements, such as cables (not shown in FIG. 12), extending within the catheter member 1220 and operably coupled to the four control element interface assemblies 1215a-d, such that operation of the interface assemblies 1215a-d bends the distal end of the catheter member 1220 in four separate directions, e.g., by displacing one of the control elements in the proximal direction to deflect the distal end of the catheter member 1220 in the predetermined direction dictated by the one control element, while allowing the other three control elements to be displaced in the distal direction as a natural consequence of the catheter member deflect. Further details discussing the structure of the sheath 301, guide catheter 302 and routing of control elements therein are described in further detail in various applications that have previously been incorporated by reference.

From a functional perspective, in most embodiments the sheath 301 need not be as drivable or controllable as the associated guide instrument 302, because the sheath instrument 301 is generally used to contribute to the remote tissue access schema by providing a conduit for the guide instrument 302, and to generally point the guide catheter member 1220 in the correct direction. Such movement is controlled by rolling the sheath catheter member 1120 relative to the patient and bending the sheath catheter member 1220 in one or more directions with the control element.

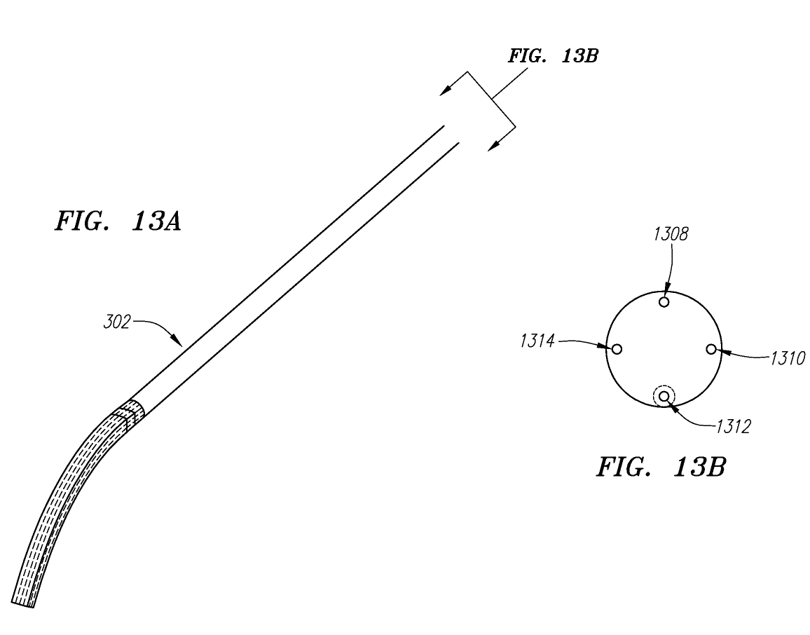

FIGS. 13A-16B further illustrate the basic kinematics of a guide catheter 301 with four independently controllable control elements 1308, 1310, 1312, 1314, such as wires, the manipulation of which is governed by an adjusted control model 116 according to one embodiment.

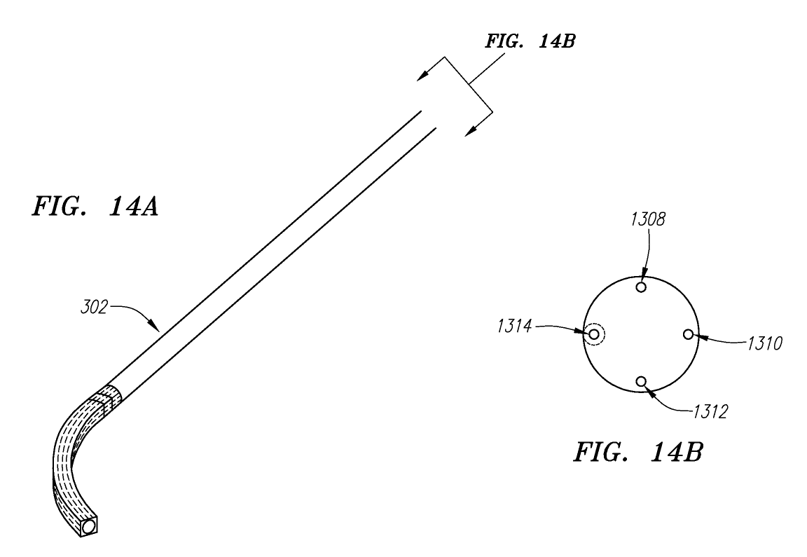

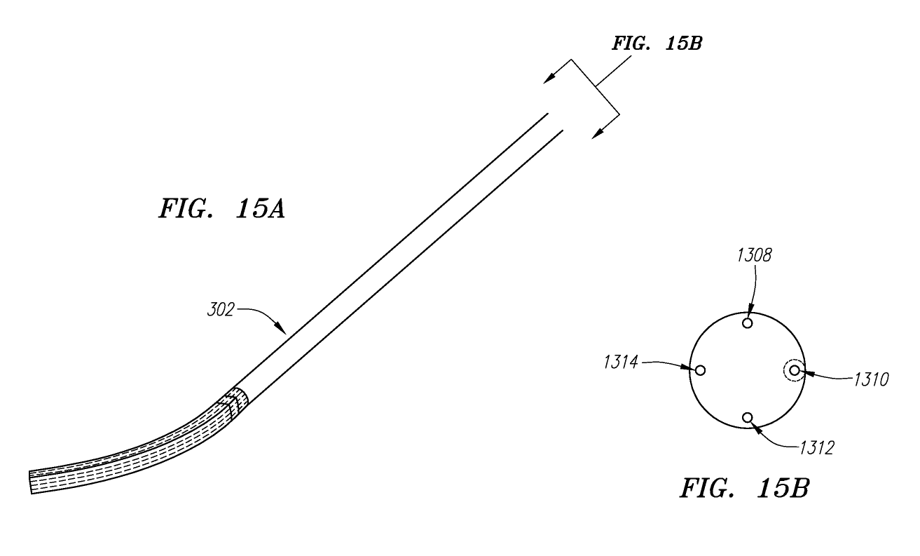

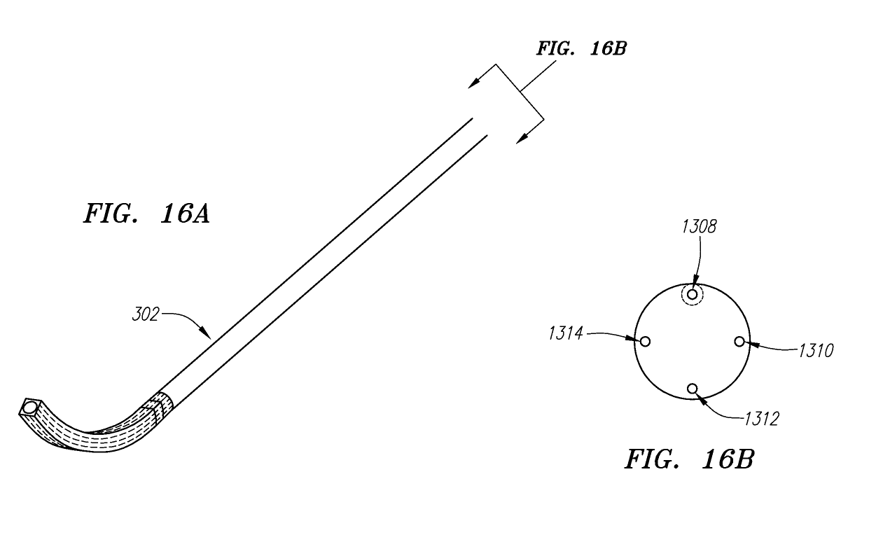

Referring to FIGS. 13A-B, as tension is placed only upon the bottom control element 1312, the guide catheter 302 bends downwardly, as shown in FIG. 13B. Similarly, pulling the left control element 1314 in FIGS. 14A-B bends the catheter 302 left, pulling the right control element 1310 in FIGS. 15A-B bends the catheter 302 right, and pulling the top control element 1308 in FIGS. 16A-B bends the catheter 302 upwardly. As will be apparent to those skilled in the art, well-known combinations of applied tension about the various control elements results in a variety of bending configurations at the tip of the guide catheter 302. One of the challenges in accurately controlling a catheter or similar elongate member with tension control elements is the retention of tension in control elements, which may not be the subject of the majority of the tension loading applied in a particular desired bending configuration. If a system or instrument is controlled with various levels of tension, then losing tension, or having a control element in a slack configuration, can result in an unfavorable control scenario. Similar control can be implemented using other numbers of control elements, e.g., two control elements for bending motion in opposite directions, and three control elements.

FIGS. 17-34 further illustrate a kinematics control model that may be utilized to controllably manipulate a guide catheter 302, and which may be adjusted 114 such that the guide catheter 302 is controlled according to an adjusted control model 116 to account for the particular working instrument 130 or ablation catheter that is inserted into the working lumen of the guide catheter 302.

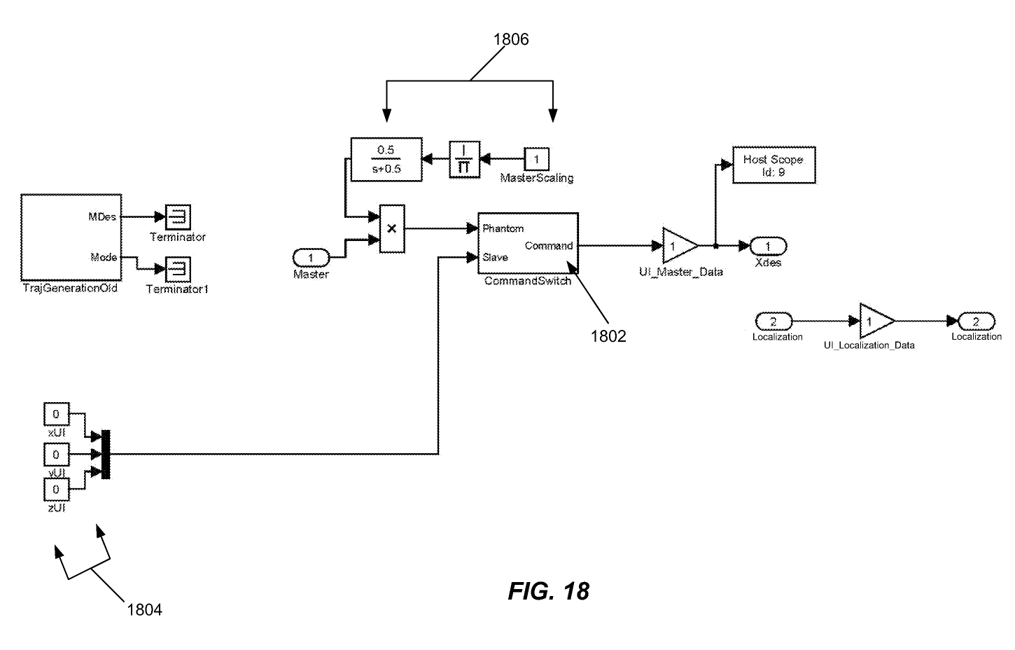

In one system (S), referring to FIG. 17, inputs to functional block 1701 are XYZ position of the master input device 324 in the coordinate system of the master input device 324 which, per a setting in the software of the master input device 324 may be aligned to have the same coordinate system as the guide catheter 302, and localization XYZ position of the distal tip of the instrument as measured by the localization system in the same coordinate system as the master input device 324 and catheter 302. Referring to FIG. 18, for a more detailed view of functional block 1701 of FIG. 17, a switch 1802 is provided at block to allow switching between master inputs for desired catheter 302 position, to an input interface 1804 through which an operator may command that the instrument go to a particular XYZ location in space. Various controls features may also utilize this interface to provide an operator with, for example, a menu of destinations to which the system should automatically drive an instrument, etc. Also depicted in FIG. 18 is a master scaling functional block 1806, which is utilized to scale the inputs coming from the master input device 324 with a ratio selectable by the operator. The command switch 1802 functionality includes a low pass filter to weight commands switching between the master input device and the input interface 1804, to ensure a smooth transition between these modes.

Referring back to FIG. 17, desired position data in XYZ terms is passed to the inverse kinematics block 1702 for conversion to pitch, yaw, and extension (or “insertion”) terms in accordance with the predicted mechanics of materials relationships inherent in the mechanical design of the guide catheter 302 instrument. The kinematic relationships for many catheters 302 may be modeled by applying conventional mechanics relationships. In summary, a control-element-steered catheter 302 is controlled through a set of actuated inputs. In a four-control-element catheter 302, for example, there are two degrees of motion actuation, pitch and yaw, which both have + and − directions. Other motorized tension relationships may drive other instruments, active tensioning, or insertion or roll of the catheter instrument 302. The relationship between actuated inputs and the catheter's 302 end point position as a function of the actuated inputs is referred to as the “kinematics” of the catheter 302.

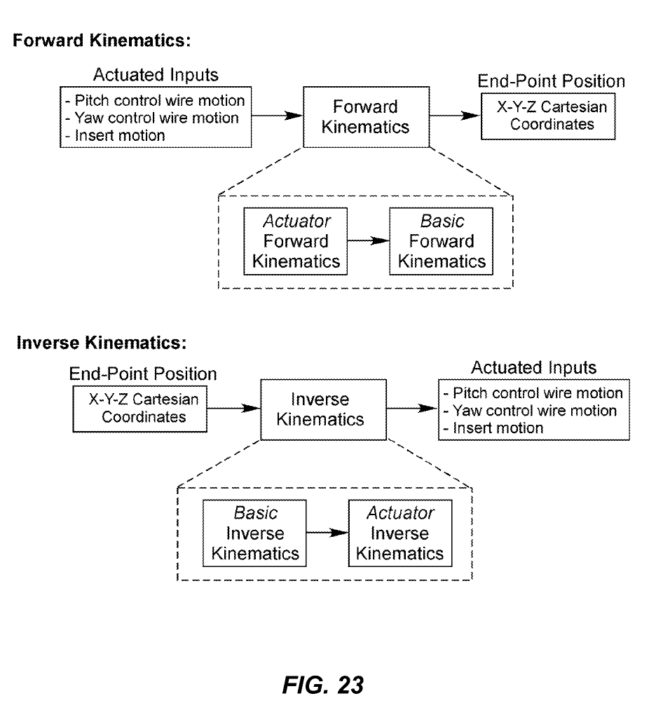

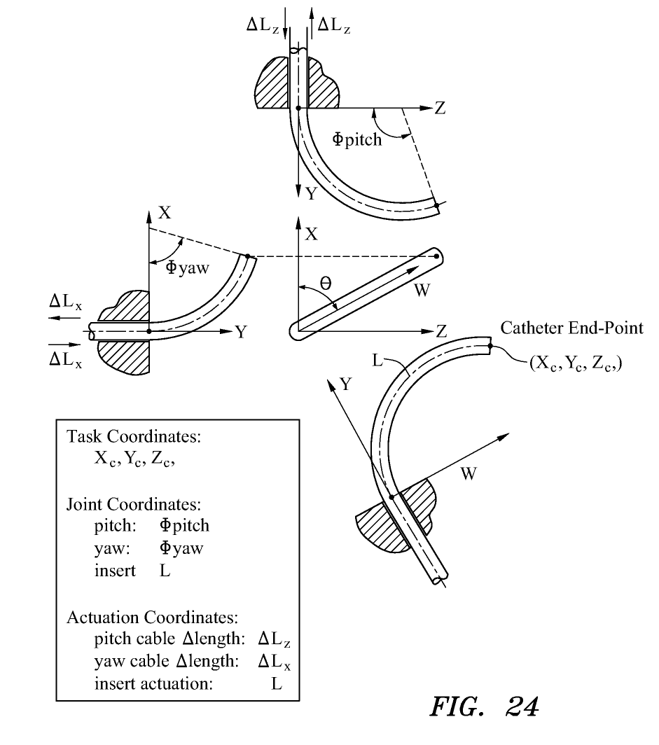

Referring to FIG. 23, the “forward kinematics” expresses the catheter's 302 end-point position as a function of the actuated inputs while the “inverse kinematics” expresses the actuated inputs as a function of the desired end-point position. Accurate mathematical models of the forward and inverse kinematics are essential for the control of a robotically controlled catheter system. For clarity, the kinematics equations are further refined to separate out common elements, as shown in FIG. 23. The basic kinematics describes the relationship between the task coordinates and the joint coordinates. In such case, the task coordinates refer to the position of the catheter end-point while the joint coordinates refer to the bending (pitch and yaw, for example) and length of the active catheter. The actuator kinematics describes the relationship between the actuation coordinates and the joint coordinates. The task, joint, and bending actuation coordinates for the robotic catheter are illustrated in FIG. 24. By describing the kinematics in this way we can separate out the kinematics associated with the catheter structure, namely the basic kinematics, from those associated with the actuation methodology.

The development of the catheter's 302 kinematics model is derived using a few essential assumptions. Included are assumptions that the catheter 302 structure is approximated as a simple beam in bending from a mechanics perspective, and that control elements, such as thin tension wires, remain at a fixed distance from the neutral axis and thus impart a uniform moment along the length of the catheter 302.

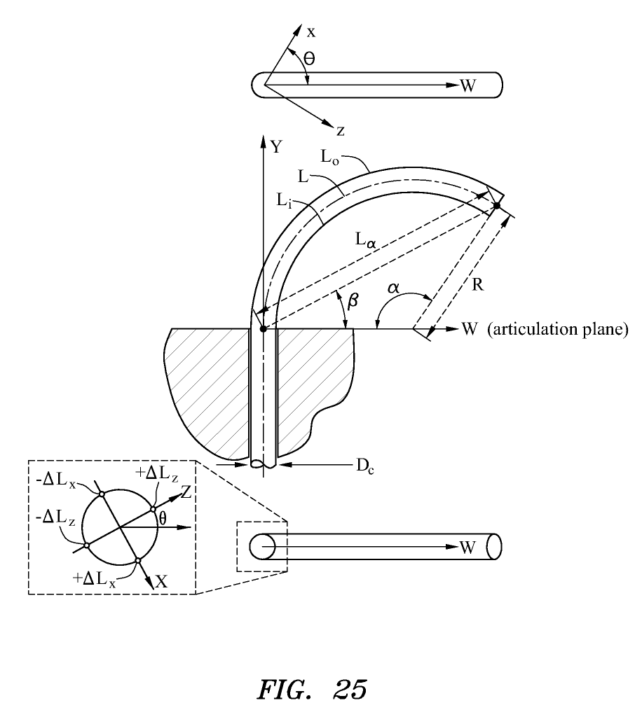

In addition to the above assumptions, the geometry and variables shown in FIG. 25 are used in the derivation of the forward and inverse kinematics. The basic forward kinematics, relating the catheter task coordinates (Xc, Yc, Zc) to the joint coordinates φ_{pitch}, φ_{yaw}, L) is given as follows:

X_{c}=w cos(θ)

Y_{c}=R sin(α)

Z_{c}=w sin(θ)

where

$$w = R ( 1 - cos ( α ) ) \\ \begin{matrix}α = {[ {( {φ}_{pitch} )}^{2} + {( {φ}_{yaw} )}^{2} ]}^{1 / 2} & ( total bending ) \\ R = \frac{L}{α} & ( bend radius ) \\ θ = atan 2 ( {φ}_{pitch} , {φ}_{yaw} ) & ( roll angle )\end{matrix} \qquad$$

wherein α is a total bending, R is a bend radius, and θ is a roll angle, respectively, of the bending portion of the guide instrument, and actuator forward kinematics, relating the joint coordinates, φ_{pitch}, φ_{pitch}, L, to actuator coordinates, ΔLₓ, ΔL_{z}, L, is expressed as follows:

$${φ}_{pitch} = \frac{2 Δ {L}_{z}}{{D}_{c}}$$

$${φ}_{yaw} = \frac{2 Δ {L}_{x}}{{D}_{c}} .$$

The actuator forward kinematics, relating the joint coordinates (φ_{pitch}, φ_{pitch}, L) to the actuator coordinates (ΔLₓ, ΔL_{z}, L) is given as follows:

$${φ}_{pitch} = \frac{2 Δ {L}_{z}}{{D}_{c}}$$

$${φ}_{yaw} = \frac{2 Δ {L}_{x}}{{D}_{c}} .$$

As illustrated in FIG. 23, the catheter's end-point position can be predicted given the joint or actuation coordinates by using the forward kinematics equations described above. Calculation of the catheter's actuated inputs as a function of end-point position, referred to as the inverse kinematics, can be performed numerically, using a nonlinear equation solver such as Newton-Raphson. A more desirable approach, and the one used in this illustrative embodiment, is to develop a closed-form solution which can be used to calculate the required actuated inputs directly from the desired end-point positions.

As with the forward kinematics, we separate the inverse kinematics into the basic inverse kinematics, which relates joint coordinates to the task coordinates, and the actuation inverse kinematics, which relates the actuation coordinates to the joint coordinates. The basic inverse kinematics, relating the joint coordinates (φ_{pitch}, φ_{pitch}, L), to the catheter task coordinates (Xc, Yc, Zc) is given as follows:

φ_{pitch}=α sin(θ)

φ_{yaw}=α cos(θ)

L−Rα

$$\to where \to \to \begin{matrix}\vec{θ = atan 2 ( {Z}_{c} , {X}_{c} )} \\ R = \frac{l sin β}{sin 2 β} \\ α = π - 2 β\end{matrix} \to \begin{matrix}\overline{β = atan 2 ( {Y}_{c} , {W}_{c} )} \\ {W}_{c} = {( {X}_{c}^{2} + {Z}_{c}^{2} )}^{1 / 2} \\ \underline{l = {( {W}_{c}^{2} + {Y}_{c}^{2} )}^{1 / 2}}\end{matrix}$$

The actuator inverse kinematics, relating the actuator coordinates (ΔLₓ, ΔL_{z}, L) to the joint coordinates (φ_{pitch}, φ_{pitch}, L) is given as follows

$$Δ {L}_{x} = \frac{{D}_{c} {φ}_{yaw}}{2}$$

$$Δ {L}_{z} = \frac{{D}_{c} {φ}_{pitch}}{2}$$

where

α=total bending of a bending portion of the guide instrument,

R=bend radius of the bending portion of the guide instrument,

Θ=roll angle of the bending portion of the guide instrument,

L=length of the guide extension out the distal end of the sheath

β=an intermediate variable

Wc=another intermediate variable, projection of the length of the catheter onto the XZ plane

l=a further intermediate variable

Dc=physical diameter of the catheter

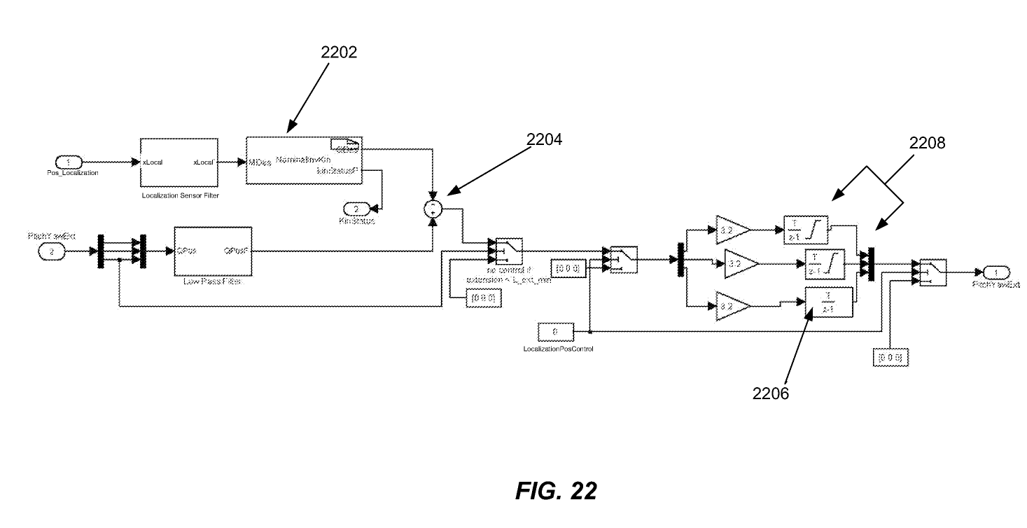

Referring back to FIG. 17, pitch, yaw, and extension commands are passed from the inverse kinematics 1702 to a position control block 1704 along with measured localization data. FIG. 22 provides a more detailed view of the position control block 1704. After measured XYZ position data comes in from the localization system, it goes through an inverse kinematics block 2202 to calculate the pitch, yaw, and extension the instrument needs to have in order to travel to where it needs to be. Comparing 2204 these values with filtered desired pitch, yaw, and extension data from the master input device, integral compensation is then conducted with limits on pitch and yaw to integrate away the error. In this embodiment, the extension variable does not have the same limits 2206, as do pitch and yaw 2208. As will be apparent to those skilled in the art, having an integrator in a negative feedback loop forces the error to zero. Desired pitch, yaw, and extension commands are next passed through a catheter workspace limitation 1706 (FIG. 17), which may be a function of the experimentally determined physical limits of the instrument beyond which componentry may fail, deform undesirably, or perform unpredictably or undesirably. This workspace limitation essentially defines a volume similar to a cardioid-shaped volume about the distal end of the instrument. Desired pitch, yaw, and extension commands, limited by the workspace limitation block, are then passed to a catheter roll correction block 1708 (FIG. 17).

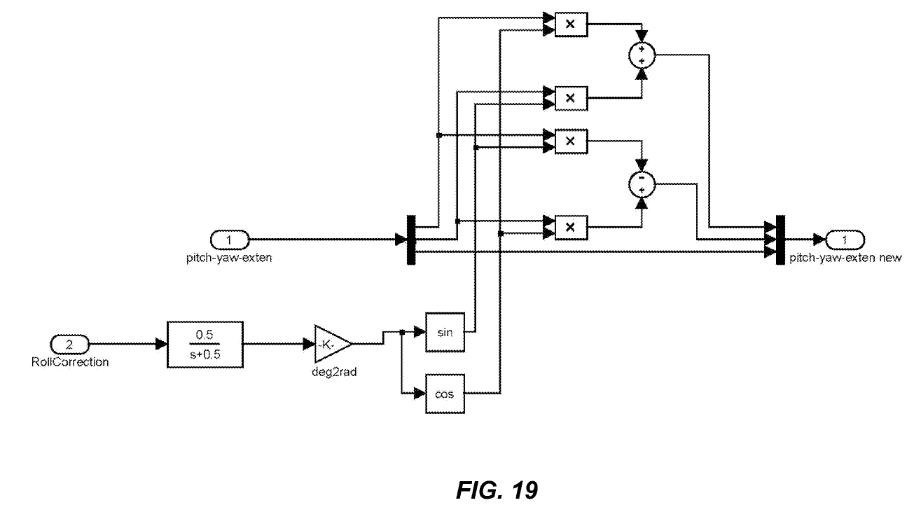

This functional block is depicted in further detail in FIG. 19, and essentially comprises a rotation matrix for transforming the pitch, yaw, and extension commands about the longitudinal, or “roll”, axis of the instrument—to calibrate the control system for rotational deflection at the distal tip of the catheter that may change the control element steering dynamics. For example, if a catheter has no rotational deflection, pulling on a control element located directly up at twelve o'clock should urge the distal tip of the instrument upward. If, however, the distal tip of the catheter has been rotationally deflected by, say, ninety degrees clockwise, to get an upward response from the catheter, it may be necessary to tension the control element that was originally positioned at a nine o'clock position. The catheter roll correction schema depicted in FIG. 18 provides a means for using a rotation matrix to make such a transformation, subject to a roll correction angle, such as the ninety degrees in the above example, which is input, passed through a low pass filter, turned to radians, and put through rotation matrix calculations.

In one embodiment, the roll correction angle is determined through experimental experience with a particular instrument and path of navigation. In another embodiment, the roll correction angle may be determined experimentally in-situ using the accurate orientation data available from the preferred localization systems. In other words, with such an embodiment, a command to, for example, bend straight up can be executed, and a localization system can be utilized to determine at which angle the defection actually went—to simply determine the in-situ roll correction angle.

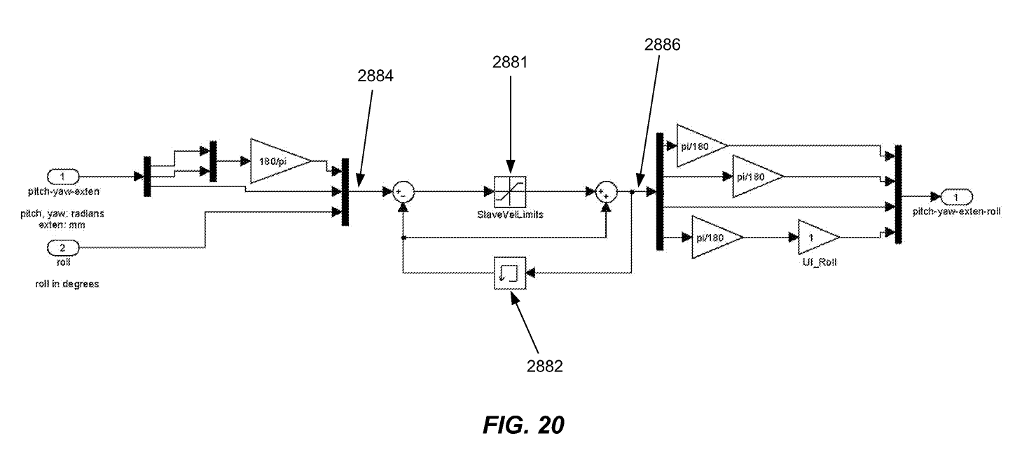

Referring briefly back to FIG. 17, roll corrected pitch and yaw commands, as well as unaffected extension commands, are output from the roll correction block 1708 and may optionally be passed to a conventional velocity limitation block 1710. Referring to FIG. 20, pitch and yaw commands are converted from radians to degrees, and automatically controlled roll may enter the controls picture to complete the current desired position from the last servo cycle. Velocity is calculated by comparing the desired position from the previous servo cycle 2001, as calculated with a conventional memory block 2002 calculation, with that of the incoming commanded cycle. A conventional saturation block 2004 keeps the calculated velocity within specified values, and the velocity-limited command 2006 is converted back to radians and passed to a tension control block 1712 (FIG. 17).

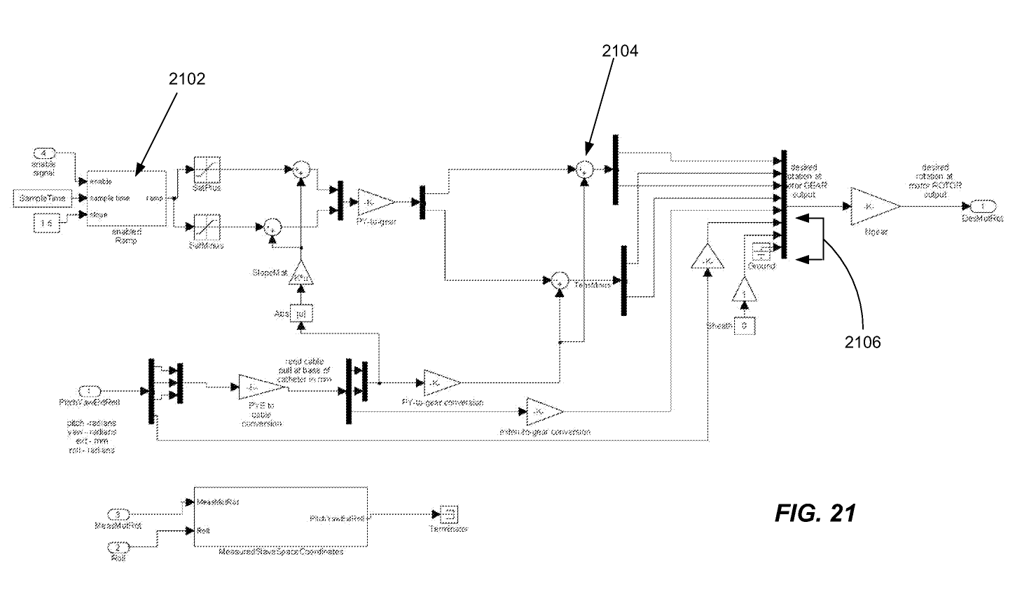

Tension within control elements may be managed depending upon the particular instrument embodiment, as described above in reference to the various instrument embodiments and tension control mechanisms. As an example, FIG. 21 depicts a pre-tensioning block 2102 with which a given control element tension is ramped to a present value. An adjustment is then added to the original pre-tensioning based upon a preferably experimentally-tuned matrix pertinent to variables, such as the failure limits of the instrument construct and the incoming velocity-limited pitch, yaw, extension, and roll commands. This adjusted value is then added 2104 to the original signal for output, via gear ratio adjustment, to calculate desired motor rotation commands for the various motors involved with the instrument movement. In this embodiment, extension, roll, and sheath instrument actuation 2106 have no pre-tensioning algorithms associated with their control. The output is then complete from the master following mode functionality, and this output is passed to a primary servo loop. Additional details regarding these components and their operation are described in U.S. application Ser. No. 11/073,363, filed Mar. 4, 2005, the contents of which were previously incorporated herein by reference.

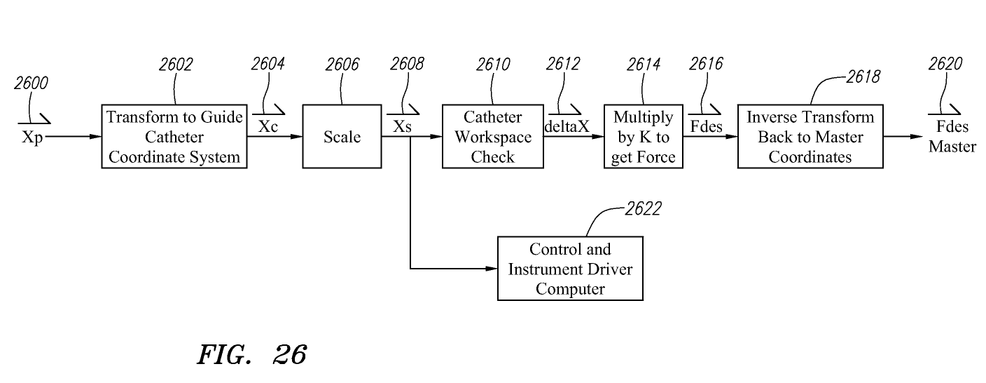

Referring to FIG. 26, a sample flowchart of a series of operations leading from a position vector applied at the master input device to a haptic signal applied back at the operator is depicted. A vector 2600 associated with a master input device move by an operator may be transformed into an instrument coordinate system, and in particular to a catheter instrument tip coordinate system, using a simple matrix transformation 2602. The transformed vector 2604 may then be scaled 2606 per the preferences of the operator, to produce a scaled-transformed vector 2608. The scaled-transformed vector 2608 may be sent to both the control and instrument driver computer 2622 preferably via a serial wired connection, and to the master computer for a catheter workspace check 2610 and any associated vector modification 2612 this is followed by a feedback constant multiplication 2614 chosen to produce preferred levels of feedback, such as force, in order to produce a desired force vector 2616, and an inverse transform 2618 back to the master input device coordinate system for associated haptic signaling to the operator in, that coordinate system 2620.

A conventional Jacobian may be utilized to convert a desired force vector 2616 to torques desirably applied at the various motors comprising the master input device, to give the operator a desired signal pattern at the master input device. Given this embodiment of a suitable signal and execution pathway, feedback to the operator in the form of haptics, or touch sensations, may be utilized in various ways to provide added safety and instinctiveness to the navigation features of the system, as discussed in further detail below.

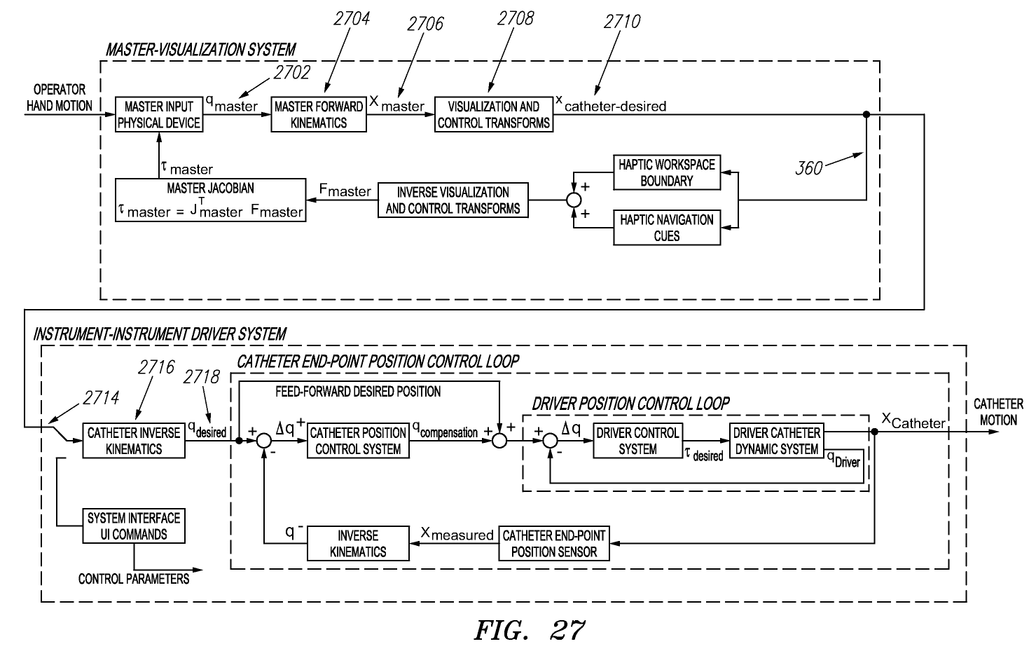

FIG. 27 is a system block diagram including haptics capability. As shown in summary form in FIG. 27, encoder positions on the master input device, changing in response to motion at the master input device, are measured 2702, sent through forward kinematics calculations 2704 pertinent to the master input device to get XYZ spatial positions of the device in the master input device coordinate system 2706, then transformed 2708 to switch into the catheter coordinate system and (perhaps) transform for visualization orientation and preferred controls orientation, to facilitate “instinctive driving.”

The transformed desired instrument position 2710 may then be sent down one or more controls pathways to, for example, provide haptic feedback 2712 regarding workspace boundaries or navigation issues, and provide a catheter instrument position control loop 2714 with requisite catheter desired position values, as transformed utilizing inverse kinematics relationships for the particular instrument 2716 into yaw, pitch, and extension, or “insertion”, terms 2718 pertinent to operating the particular catheter instrument with open or closed loop control.

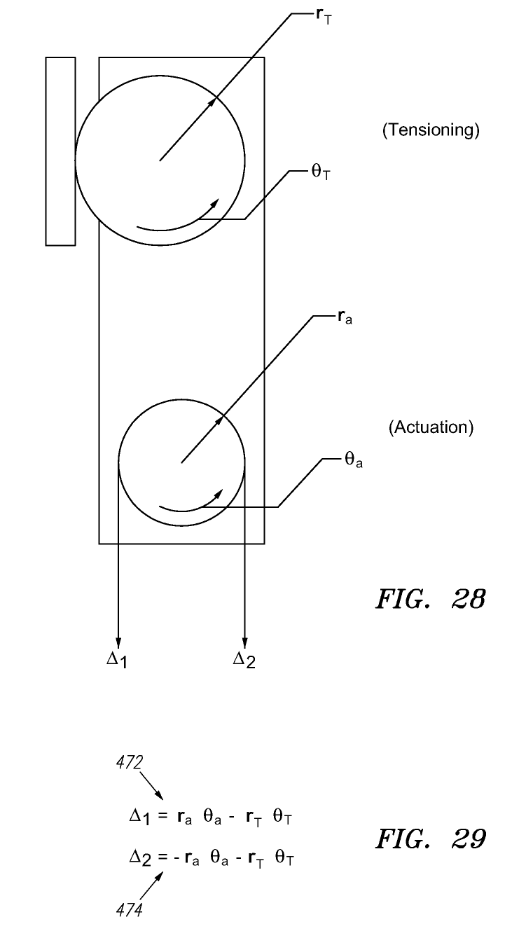

Referring to FIGS. 28-33, relationships pertinent to tension control, e.g., via a split carriage design such as that depicted in U.S. application Ser. No. 11/073,363, filed Mar. 4, 2005, the contents of which were previously incorporated herein by reference, and which is a design that may isolate tension control from actuation for each associated degree of freedom, such as pitch or yaw of a steerable catheter instrument.

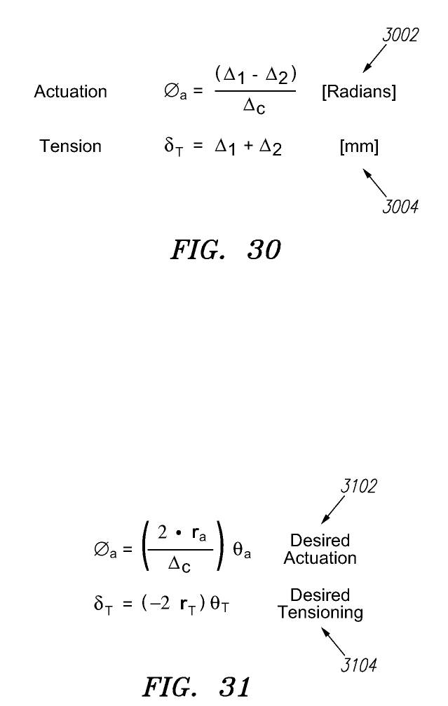

Referring to FIG. 28, some of the structures associated with a split carriage design, include a linearly movable portion, a guide instrument interface socket, a gear, and a rack. Applying conventional geometric relationships to the physical state of the structures related in FIG. 28, the equations 2001, 2004 of FIG. 20 may be generated. Utilizing forward kinematics of the instrument, such as those described above in reference to a pure cantilever bending model for a catheter instrument, the relationships of FIG. 30 may be developed for the amount of bending as a function of cable pull and catheter diameter (“Dc”) 3002, and for tension 3004, defined as the total amount of common pull in the control elements. Combining the equations of FIG. 29 and FIG. 30, one arrives at the relationships 3102, 3104 depicted in FIG. 31, wherein desired actuation and desired tensioning are decoupled by the mechanics of the involved structures. Desired actuation 3102 of the guide instrument interface socket depicted in FIG. 28 is a function of the socket's angular rotational position. Desired tensioning 3104 of the associated control elements is a function of the position of the tensioning gear versus the rack.

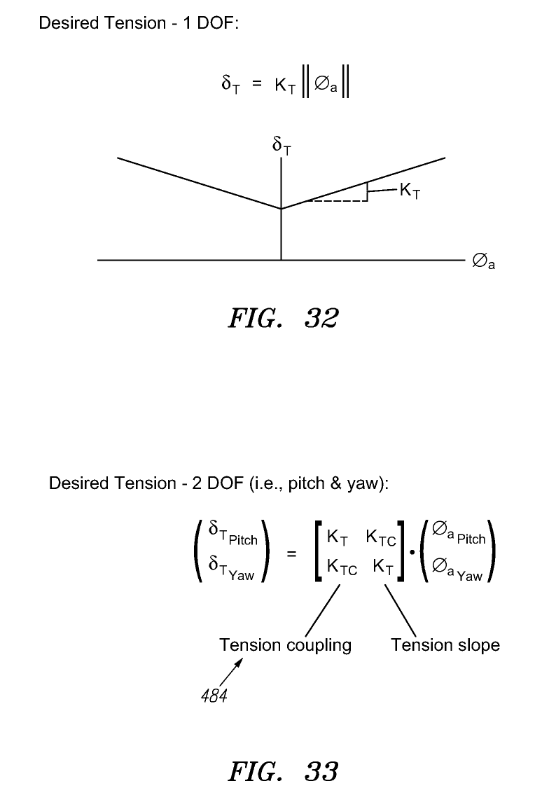

Referring to FIG. 32, with a single degree of freedom actuated, such as .+−. pitch or .+−. yaw, and active tensioning via a split carriage mechanism, desired tension is linearly related to the absolute value of the amount of bending, as one would predict. The prescribed system never goes into slack—desired tension is always positive, as shown in FIG. 33. A similar relationship applies for a two degree of freedom system with active tensioning—such as a four-cable system with .+−. pitch and .+−. yaw as the active degrees of freedom and active tensioning via a split carriage design. Since there are two dimensions, coupling terms are incorporated to handle heuristic adjustments to, for example, minimize control element slacking and total instrument compression.

Having described in detail an example of a system (S) in which embodiments may be implemented and a kinematics control model 112 for use in the system (S), FIGS. 34-39 illustrate embodiments that may be implemented in the system and utilizing the kinematics control model as described above in detail.

Referring to FIG. 34, according to another embodiment, a method 3400 of adjusting the manner in which a robotic surgical system (S) operates includes inserting a working instrument 130 into a working lumen of a guide catheter 302 at step 3405. In the illustrated system (S) example, the guide catheter 302 is coaxial with a sheath 301. According to one embodiment, the working instrument 130 is an ablation catheter, but other working instruments including, but not limited to, a biopsy forceps, a dilator and a needle may be utilized.

At step 3410, data 132 is acquired or read from the memory device 131 attached to the working instrument 130, e.g., by a controller 110 or other associated control component. In another embodiment, the data 132 may be acquired or read from an external data source associated with the working instrument 130, or manually entered by an operator. Thus, the data 132 may be acquired directly from the working instrument 130 (via an attached storage device 131), or independently. of the working instrument 130. The data 132 may be mechanical data 3412 (e.g., stiffness or modulus, mechanical impedance, friction, etc.) and/or physical data 3414 (e.g. dimensions, outer diameter, length, etc.).

At step 3415, the control model 112, e.g., the kinematics model as described above, is automatically adjusted based on the working instrument data 132. As discussed above, one example of a kinematics control model 112 that can be used in embodiments predicts a spatial position of a bending portion of the guide instrument 132, X_{c}, Y_{c}, Z_{c}, utilizing joint coordinates, φ_{pitch}, φ_{yaw}, L, and may determine actuated inputs for controlling the at least one control element based on a desired position of a bending portion of the guide instrument, X_{c}, Y_{c}, Z_{c}, utilizing joint coordinates, φ_{pitch}, φ_{yaw}, L.

Various aspects or coefficients may be adjusted 114 (increased or decreased) as necessary to adapt 114 the control model 112 to the particular mechanical and/or physical attributes of the working instrument 130 and account for the particular working instrument 130 that is employed. In another embodiment, a variable may be deleted (which amounts to a “0”) coefficient. Further, according to one embodiment, a forward kinematics model is adjusted. In another embodiment, an inverse kinematics model is adjusted.

Adjustments 114 to the kinematics control model 112 may involve adjustment 3417 to a control model 112 for the guide catheter 302 which, in turn, adjust one or more servo motors of the instrument driver 305 in order to adjust the manner in which a distal bending portion of the guide catheter 302 is manipulated. Adjustments 114 to the kinematics control model 112 may also involve adjustment 3419 to a control model 112 for the sheath instrument 301 in order to which, in turn, adjust one or more servo motors of the instrument driver 305 in order to adjust the manner in which a distal bending portion of the sheath instrument 301 is manipulated. In other embodiments, multiple control models 112(1-n) (e.g., of both the sheath 301 and the guide 302) may be adjusted as necessary.

Further, control model adjustments may involve axial stiffness. In another embodiment, the control model adjustments involve bending stiffness. The adjustments may also involve a combination of both. Other adjustments may involve a feed-forward term wherein the coefficient of friction between the working catheter and an inner surface of the guide instrument is estimated. These attributes can also be measured on a test bench (e.g., with saline infusion, etc.) and entered into a lookup table.

As a further example, in another embodiment, the control adjustment may be an adjustment to a catheter stiffness coefficient, e.g., represented as a matrix, e.g., a “Km matrix” in the matrix expression Km q=Gτ wherein Kₘ is a stiffness matrix for a working instrument, G is the geometry describing distributed moments and axial directed tension, and τ is a tension vector, as described in further detail in U.S. App. No. 60/898,661 and Ser. No. 12/022,987 (Docket No. 20032.00), the contents of which are incorporated herein by reference. A control model 112 that may be adjusted or adapted to account for the particular working instrument 130 employed is based on the matrix expression Km q=Gτ, wherein Kₘ is the stiffness matrix, which may be adjusted to account for the particular working catheter employed, and is expressed as follows:

$$Δ {l}_{t} = {l}_{0} ( {G}^{T} + \frac{1}{{K}_{t}} {G}^{†} {K}_{m} ) q . \qquad ( 47 )$$

This mechanics model specifies how a mechanics model input in the form of a desired beam configuration (i.e., output of a kinematics model 121) may mapped to an associated displacement of a deflection member or control element, such as a pull wire, for an isolated section of the catheter. This mechanics model is also bi-directional such that the control element displacement may be mapped to the catheter shape or configuration.

In the above example control model, the Km matrix represents the bending and axial stiffness of conglomerate instrument comprising the guide and the working instrument inserted through the working lumen of the guide. Adjustments are made as necessary to adapt 114 the control model 112 to the particular mechanical and/or physical attributes of the working instrument 130.

At stage 3420, the instrument driver 305 and robotic instrument system (S) are operated using the adjusted control model(s) 112. In this manner, an unadjusted or default control model 112 does not result in under-bending or over-bending of the working instrument 130. Instead, embodiments adapt or adjust 114 the kinematics control model 112 to the particular mechanical and/or physical attributes of the specific working instrument 130 to prevent or minimize errors that may otherwise occur without adjustments provided by embodiments.

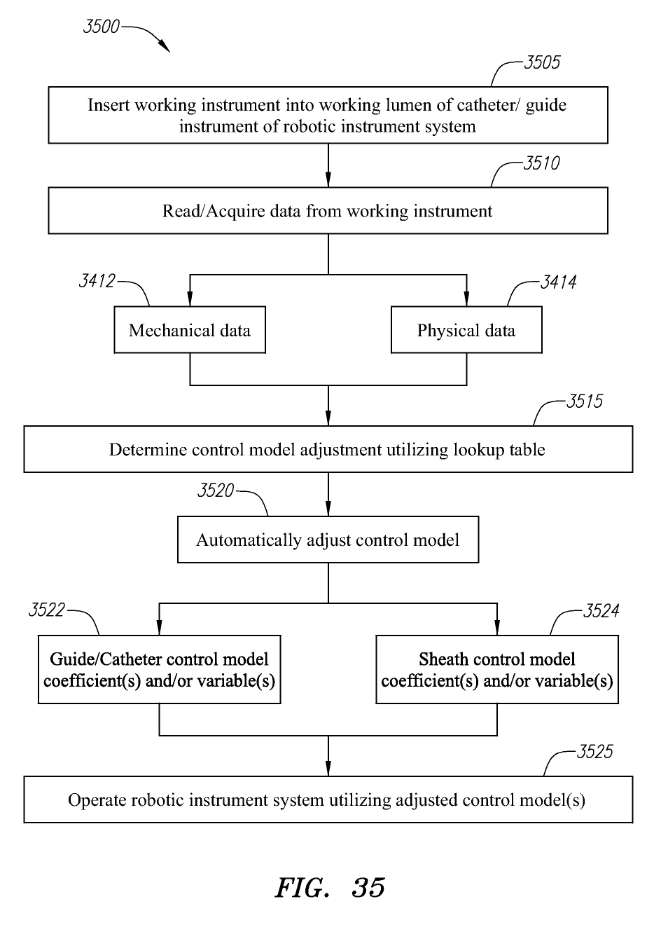

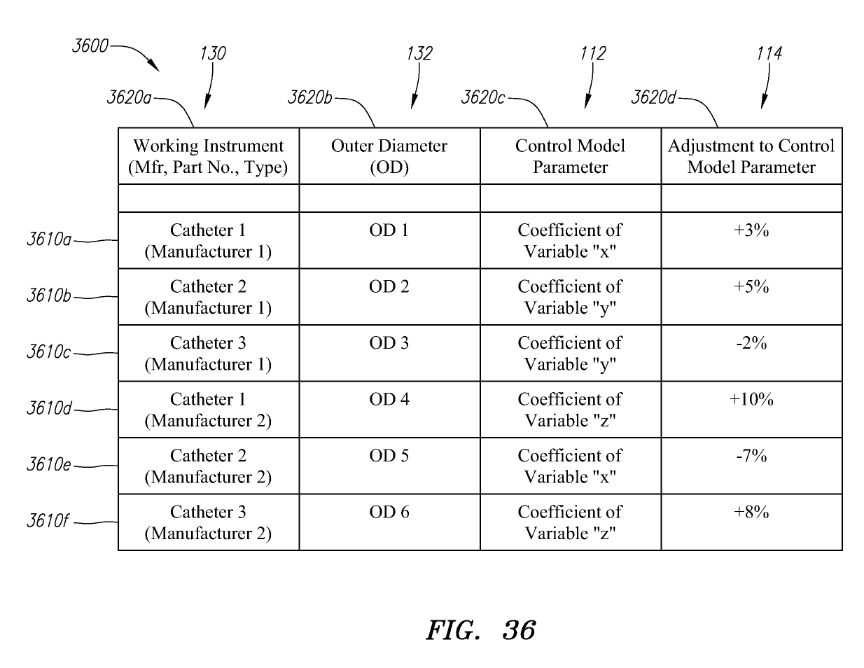

FIG. 35 illustrates another embodiment of adjusting 114 the manner in which a robotic surgical system (S) operates. As shown in FIG. 34, the method 3500 includes inserting a working instrument 130 into a working lumen of a guide catheter 302 at step 3505, reading or acquiring data 132 at step 3510, which may be mechanical data 3412 (e.g., stiffness or modulus, mechanical impedance, friction, etc.) and/or physical data 3414 (e.g. dimensions, outer diameter, length, etc.). At step 3515, the control model 112, e.g., the kinematics control model 112 as described above, is automatically adjusted 114 based on the data 132. In the illustrated embodiment, step 3515 is performed using a lookup table (examples of which are shown in FIGS. 36-39). At stage 3520, the kinematics control model 112 is automatically adjusted 114 based on the determination at stage 3515. In one embodiment, the adjustment 114 may involve adjusting 114 a coefficient and/or variable of the control model 112. In another embodiment, a coefficient and/or variable of a control model 112 of the sheath 301 is adjusted 114. In another alternative embodiment, adjustments 114 involve both the guide catheter 302 and sheath 301 control models 112 and may involve a coefficient and/or variable of respective control models 112.

Referring to FIG. 36, the data 132 may be in the form of a lookup table 3600 constructed according to one embodiment includes data regarding a physical attribute 3414, e.g., the outer diameter (OD) of the working instrument 130 in the form of a catheter such as an ablation catheter. The OD data may be used to determine or frictional forces between an outer surface of the ablation catheter 130 and an inner surface of the guide instrument 132.

The lookup table 3600 includes a plurality of rows 3610a-n and columns 3620a-n. In the illustrated embodiment, the first column 3620 identifies three different working instruments Catheters 1-3 manufactured by a first manufacturer, and three different working instruments, Catheters 1-3, manufactured by a second manufacturer. In the illustrated embodiment, each row corresponds to an individual catheter. In the illustrated example, the lookup table 3600 includes three catheters, which may be of the same or different type, and provided by the same manufacturer, and three other catheters, which may also be of the same or different type, provided by a different manufacturer. The outer diameter of each catheter is provided in column 3620. The adjustment 114 to the control model 112 that is required based on the various outer diameters is indicated in column 3620c. In the illustrated embodiment, the adjustment 114 involves changing the value of a single coefficient, but in other embodiments, and adjustment 114 may involve changing the values of multiple coefficients, changing or adding a variable, or a combination thereof. Column 3620d indicates the magnitude of the adjustment to the control model parameter indicated in column 3620c.

For example, row 3610d includes data corresponding to Catheter 1, which is manufactured by Manufacturer 2. This catheter has an outer diameter of OD5, and it is determined that the coefficient of a certain variable “z’, as an example, should be increased by 10% to compensate for the OD of this catheter. Similar adjustments are provided for other catheters of different sizes. In this manner, the OD of a catheter is one basis for adjusting 114 the control model 112, thereby resulting in a more accurate and effective surgical procedure.

Data used to populate a lookup table can be generated and entered by experimentation, i.e., inserting various working instrument through a working lumen of a guide and conducting tests to see how the working instrument can be manipulated with a given input. If, for example, working instrument 1 is driven to 90 degrees but only bends 80 degrees, then an adjustment to a control model 112 can be determined to effect an extra 10 degrees of articulation. This procedure can be repeated for a multitude of other catheters, for other types of working instruments, and may involve one or more different types of mechanical and/or physical attributes of the working instrument.

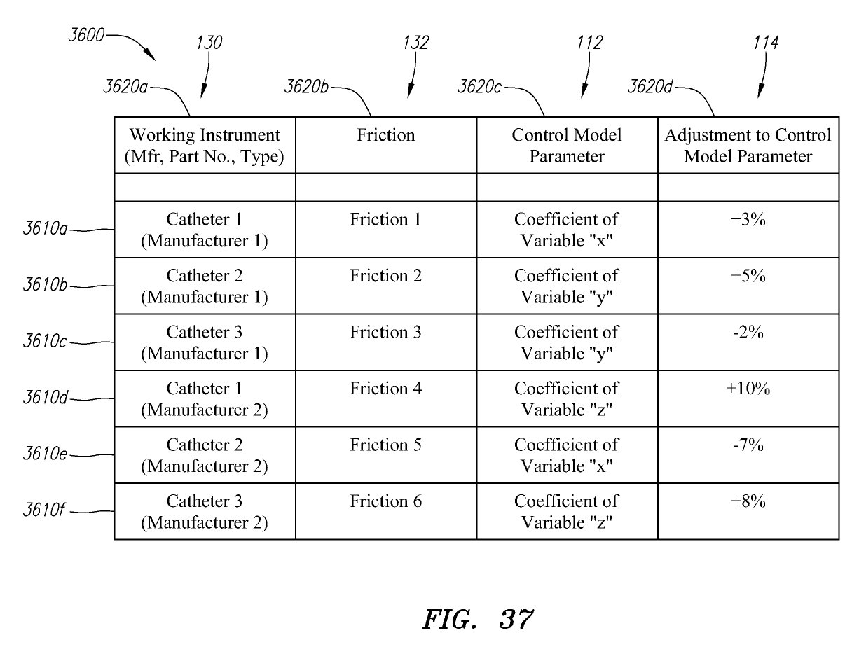

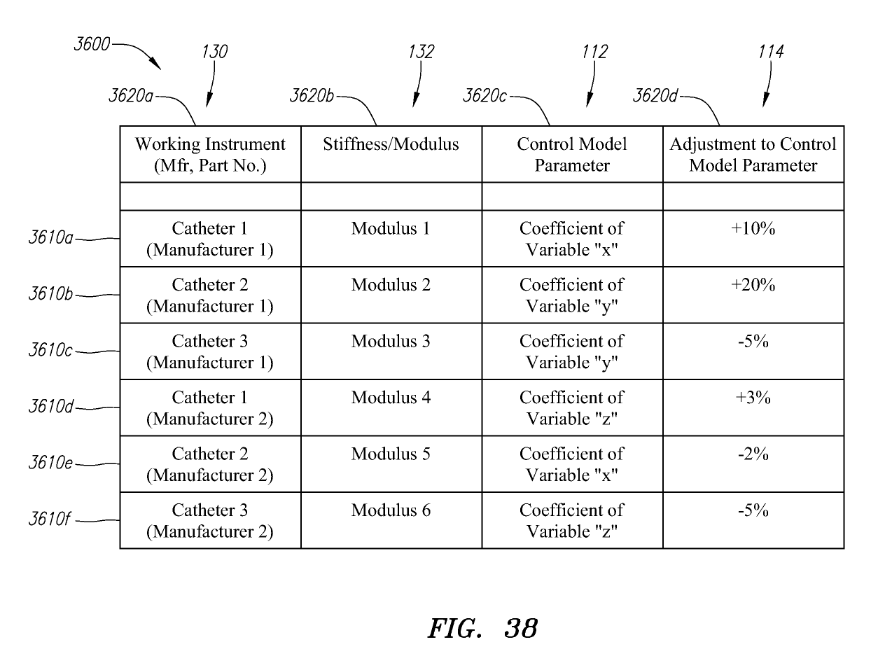

Referring to FIG. 37, in another embodiment, the data 132 is in the form of a lookup table 3700 that includes data of friction between an outer surface of the working instrument 130 and an inner surface of the guide catheter 302 is included in the lookup table. Thus, for example, when working instrument 130 is inserted within the guide catheter 302 and the data 132 is read or retrieved from the instrument 130, the friction force associated with that particular instrument can be used to adjust 114 the control model 112. For example, the catheter in row 3610e has a Friction force 5 that requires a reduction in the coefficient of variable “x” by 7%.

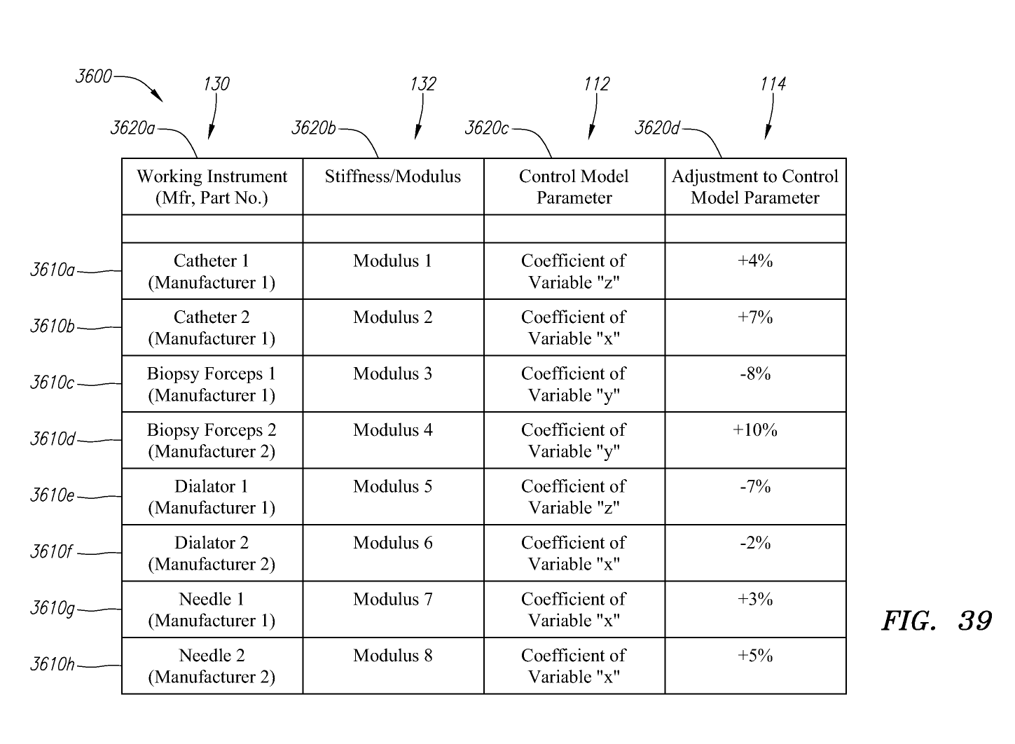

FIG. 38 illustrates a lookup table 3800 that includes data of a stiffness or modulus of various working instruments or catheters 130. FIG. 39 illustrates how coefficients can also vary with different types of working instruments, whereas FIGS. 36-38 illustrate how coefficients can vary with the same type of working instruments 130.

Although embodiments of lookup tables illustrate adjusting 114 a single coefficient of a single variable, other embodiments may involve additional and more complex adjustments, e.g., adjusting two, three or other numbers of variables as needed. Further, although embodiments are described with respect to adjusting a coefficient of a variable, other adjustments 114 may involve deleting a variable and/or adding a new variable to the control model 112. Accordingly, FIGS. 36-39 are provided as general, illustrative examples to illustrate how different mechanical and physical properties of different working instruments 130 can be used as the basis for adjusting 114 a control model to provide an adjusted or modified control model 116 that is adapted to or customized for a particular working instrument 130.

Embodiments described above involve adjustment or adaptation of a control model 112. In another embodiment, a lookup table or database may include a plurality of control models 112 (rather than adjustments thereto). For example, in one embodiment illustrated in FIGS. 40-41, a system 4000 constructed according to another embodiment is similar to the system shown in FIG. 1 except that the controller 110 includes a database 4000 of different control models. Thus, during use, a working instrument or tool 130 is inserted into the guide, and data 132 is read from the data storage device 131. The data 131 is used to select one of the control models 112 (which are already configured to account for the particular working instrument employed) in the database 4000, and the selected control model can be used to control the component 120. Similar to before, the data 132 that is used to select a control model can be a mechanical attribute and/or a physical attribute. Thus, the database 4000 configuration shown in FIG. 41 is provided to illustrate one manner in which embodiments can be implemented.

While multiple embodiments and variations of the many aspects of the invention have been disclosed and described herein, such disclosure is provided for purposes of illustration only. Many combinations and permutations of the disclosed system are useful in minimally invasive surgery, and the system is configured to be flexible. For example, although various embodiments are described with reference to mechanical and physical properties including friction, stiffness or modulus and outer diameter, other properties may also be utilized to adjust a control model 112. Such properties include, but are not limited to, shape details (e.g., taper, non-homogeneities), materials, bending coefficients, etc. Further, a control model 112 can be adjusted based only on mechanical data, only physical data, or a combination thereof. Additionally, the control model that is adjusted may be only a control model of a guide instrument or catheter, only a control model of a sheath instrument, or control models of both sheath and guide instruments.

Further, embodiments can be implemented based on adjusting a control model to account for a particular working instrument or selecting a control model from a database of a plurality of control models to account for a particular working instrument.

Moreover, although certain embodiments are described with reference to a lookup table, information that forms the basis of control model adjustments may be contained in a database. Additionally, a lookup table or database can be structured in various ways to include different types of information. Further, the adjustments that are required for a given working instrument may be determined in various ways, including based on theoretical analysis and experimental results, which are used to select system kinematics and control algorithms that improve or are ideal for controlling a given working instrument.

Additionally, although certain embodiments are described with reference to retrieving or reading data from a storage device attached to the working instrument, data may also be input to the system during setup. Further, if the subject working tool is not already in a lookup table or database, information related to the shape, etc. of such working tool may be analyzed to determine an ideal system kinematics and control model for operating such working instrument.

Data concerning a working instrument, including mechanical and physical data, may also be stored locally or remotely. The data may be readable or retrievable from a data storage device attached to or associated with a working instrument, or the data may reside in virtual databases, such as those available utilizing local or wide area networks and/or the internet.

Accordingly, embodiments are intended to cover alternatives, modifications, and equivalents that fall within the scope of the claims.

What is claimed is:

1. A robotic instrument system, comprising:
a controller configured to control actuation of at least one servo motor; and
an elongate bendable guide instrument defining a lumen and operatively coupled to, and configured to move in response to actuation of, the at least one servo motor;
wherein the controller controls movement of the guide instrument via actuation of the at least one servo motor based at least in part upon a control model, and
wherein the control model takes into account an attribute of an elongate working instrument positioned in the guide instrument lumen.

2. The system of claim 1, wherein the attribute is a mechanical or physical attribute of a portion of the working instrument positioned within a distal bending portion of the guide instrument.

3. The system of claim 1, wherein the control model takes into account one or both of a type and a size of the working instrument.

4. The system of claim 1, wherein the control model is adapted to take into account a working instrument having sections comprising differing dimensions or other physical differences.

5. The system of claim 1, wherein the attribute of the working instrument comprises a mechanical impedance, a stiffness, or a modulus.

6. The system of claim 1, wherein the control model takes into account a frictional force between an outer surface of working instrument and an inner surface of the guide instrument.

7. The system of claim 1, the control model comprising:
a forward kinematics model expressing a desired position of a distal end portion of the guide instrument as a function of actuated inputs for controlling a control element of the guide instrument; and
an inverse kinematics model expressing actuated inputs for controlling the control element of the guide instrument as a function of the desired position of the distal end portion of the guide instrument.

8. The system of claim 1, wherein the attribute of the working instrument is obtained from a data storage element attached to or associated with the working instrument.

9. A robotic instrument system, comprising:
a controller;
an instrument driver in communication with the controller, the instrument driver having a instrument interface including an instrument drive element that moves in response to control signals generated by the controller; and
an elongate flexible guide instrument having a base and a distal bending portion, the base operatively coupled to the instrument interface, the guide instrument comprising a control element having first and second end portions, the first end portion operatively coupled to the instrument drive element through the base, and the second end portion coupled to the distal bending portion, the control element being axially moveable relative to the guide instrument by movement of the instrument drive element, wherein the controller implements a desired bending of the distal bending portion of the guide instrument by selected movement of the instrument drive element based at least in part on a control model that takes into account one or both of a mechanical attribute and a physical attribute of an elongate working instrument that is positioned within the distal bending portion of the guide instrument.

10. The system of claim 9, wherein the control model comprises a kinematic model based at least in part upon a mechanical parameter of the guide instrument.

11. The system of claim 10, wherein the kinematic model is utilized by the controller to determine a movement of the instrument drive element based upon a relationship between an angular rotation of the drive element and a resulting position of the distal bending portion of the guide instrument.

12. The system of claim 9, wherein the control model takes into account one or both of a type and a size of the working instrument.

13. The system of claim 9, wherein the control model is adapted to take into account a working instrument having sections comprising differing dimensions or other physical attributes.

14. The system of claim 9, wherein the attribute of the working instrument comprises a mechanical impedance, a stiffness, or a modulus.

15. The system of claim 9, wherein the control model takes into account a frictional force between an outer surface of working instrument and an inner surface of the guide instrument.

16. The system of claim 9, wherein the mechanical attribute and/or physical attribute is obtained from a data storage element attached to or associated with the working instrument.

17. A robotically controlled medical instrument system, comprising:
a controller;
an instrument driver operatively coupled to the controller and controllable according to a control model employed by the controller;
a guide instrument operatively coupled to the instrument driver and comprising at least one wire extending there through for controllably articulating a distal bending portion of the guide instrument under control of the instrument driver, wherein the guide instrument defines a working lumen; and
a working instrument positioned in working lumen of the guide instrument and at least partially extending through the distal bending portion, wherein the controller is adapted to automatically adjust the control model based on an attribute of the working instrument.

18. The system of claim 17, wherein the attribute includes at least one of a type, a size, a mechanical impedance, a stiffness, or a modulus.

19. The system of claim 17, wherein the control model takes into account a frictional force between an outer surface of working instrument and an inner surface of the guide instrument.

20. The system of claim 17, wherein the controller is configured to obtain the attribute of the working instrument from a data storage element attached to or associated with the working instrument.
